(19) United States
(12) Patent Application Publication
(10) Pub. No.: US 2007/0191373 A1
(43) Pub. Date: Aug. 16, 2007

(54) Heterocyclic compounds for use in the treatment of viral infections

(76) Inventors: Christopher McGuigan, Cardiff (GB); Jan Balzarini, Heverlee (BE); Erik De Clerq, Lovenjoel (BE)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 10/551,569

(22) PCT Filed: Apr. 21, 2004
(86) PCT No.: PCT/GB04/01687

§ 371 Date: Oct. 13, 2006

(30) Foreign Application Priority Data
Apr. 25, 2003 (GB) 0309506.4

(51) Int. Cl. A61K 31/53

(52) U.S. Cl. 514/243; 514/247; 514/248; 514/249; 514/250; 514/252.01; 514/252.02; 514/252.12

(57) ABSTRACT

6-substituted-3-substituted-3H-furo[2,3-d]pyrimidin-2-one and 6-substituted-2-substituted-furo[2,3-d]pyrimidine novel compounds are useful in the treatment of viral infection, in particular cytomegalovirus viral infection. The substituents are independently selected from alkyl, aryl, alkenyl and alkynyl. The preferred substituent at the 6 position is alkyl.

The present invention relates to a chemical compound and to its therapeutic use in the prophylaxis and treatment of viral infection for example human herpes viruses, particularly and human cytomegalovirus (HCMV). Cytomegalovirus is the aetiological agent in CMV retinitis and other viral infections, which can cause considerable human illness and suffering.

It has previously been noted that nucleoside analogues of the structural types 1 and 2 exhibit a potent and selective antiviral effect (McGuigan et al J. Med. Chem. 1999, 42, 4479-84 and J. Med. Chem. 2000, 43, 4993-97):

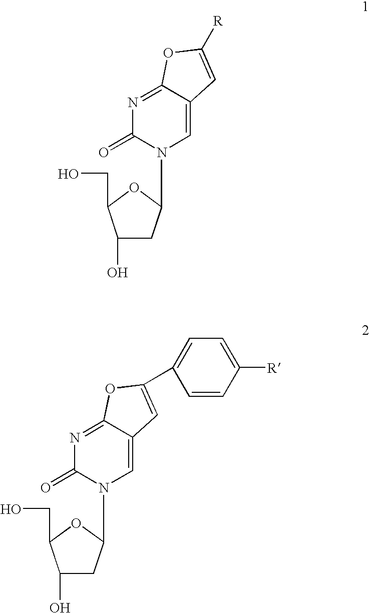

Optimal structures are 1, R═C8-C10 and 2, R=pC₅Ph. Further details are given in WO 98/49177 and WO 01/83501, respectively. The compounds exclusively inhibit Varicella zoster virus (VZV) in a VZV-thymidine-kinase dependent fashion, functioning in a classical nucleoside analogue manner, of obligate intracellular nucleoside kinase-mediated activation (Balzarini et al, Mol. Pharmacol. 61, 249-254, 2002).

It has recently been noted that dideoxynucleoside analogues of 1 have a pronounced but quite distinct activity against another member of the herpes family, namely human cytomegalovirus HCMV. The optimal structure of these agents was identified as 3 and is described in WO 01/85749.

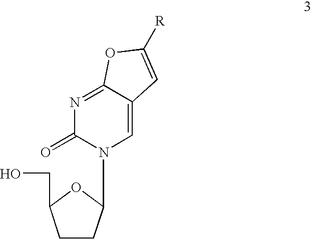

These agents would have been expected to act via a classical nucleoside mechanism, requiring 5′-phosphorylation before they would exhibit antiviral activity. As such a 5′-OH and a quasi-nucleoside structure with a sugar or close analogue was deemed necessary.

It is an object of the present invention to provide novel compounds, in particular novel compounds not requiring phosphorylation for, for example, biological activity.

It is a further object of the present invention to provide novel compounds for therapeutic use in the prophylaxis and treatment of viral infection, for example, by cytomegalovirus.

According to the present invention there is provided a chemical compound having the formula (I): 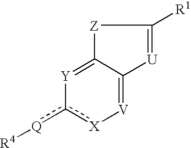
wherein:

R¹ and R⁴ are independently selected from alkyl, aryl, alkenyl and alkynyl;

Z is selected from O, NH, S, Se, NR⁵ and (CH₂)ₙ where n is 1 to 10, and CT₂ where T may be the same or different and is selected from hydrogen, alkyl and halogens, and R⁵ is alkyl, alkenyl or aryl;

Y is selected from N, CH and CR⁶ where R⁶ is alkyl, alkenyl, alkynyl or aryl;

Q is selected from O, S, NH, N-alkyl, CH₂, CHalkyl and C(alkyl)₂;

U is selected from N and CR² where R² is selected from hydrogen, alkyl, halogens, amino, alkylamino, dialkylamino, nitro, cyano, alkoxy, aryloxy, thiol, alkylthiol, arylthiol and aryl;

V is selected from N and CR³ where R³ is selected from hydrogen, alkyl, halogens, alkyloxy, aryloxy and aryl;

and when a double bond exists between X and the ring atom to which Q is attached and Q is linked to the ring moiety by a single bond, X is selected from N, CH and CR⁷, where R⁷ is selected from alkyl, alkenyl, alkynyl and aryl; and

when a double bond links Q to the ring moiety and a single bond exists between X and the ring atom to which Q is attached, R⁴ does not exist and X is NR⁸, where R⁸ is alkyl, alkenyl, alkynyl or aryl, except that when Y is N, U is CR² and V is CR³, R⁸ is not an alkyl or alkenyl group substituted at the fourth atom of the chain of said alkyl or alkenyl group, counted along the shortest route away from the ring moiety including any hetero atom present in said chain, by a member selected from OH, phosphate, diphosphate, triphosphate, phosphonate, diphosphonate, triphosphonate and pharmacologically acceptable salts, derivatives and prodrugs thereof;

and pharmacologically acceptable salts, derivatives and prodrugs of compounds of formula (I).

Surprisingly the dideoxysugar in prior art compounds known from WO 01/85749 (structure 3 above) can be replaced by an alkyl, alkenyl, alkynyl or aryl moiety that does not require phosphorylation for biological activity and hence does not require the hydroxy or any groups on the, for example, alkyl C₄ atom deemed necessary for phosphorylation.

Preferably neither R⁴ nor R⁸ contains any suitable hydroxy group that may be subject to biological phosphorylation. In particular, preferably neither R⁴ nor R⁸ is a ribose, deoxyribase, dideoxyribose, dideoxydidehydribose sugar or similar sugar group or close analogue.

Compounds having a double bond between X and the ring atom to which Q is attached are isomers of compounds having a single bond between X and the ring atom to which Q is attached. Compounds having a double bond between X and the ring atom to which Q is attached are entirely non-nucleosidic in nature. Examples of these two isomers are, for instance, structures 4 and 5:

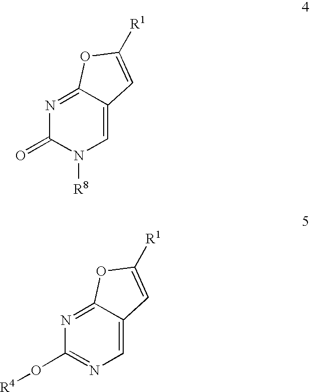

Varying the composition of R¹, R⁴ and R⁸ of formula (I) determines the biological activity of the compounds.

Preferably Z is O or NH. Where Z is N-alkyl, suitably the alkyl is C₁ to C₅ alkyl.

Preferably Y is N.

Preferably Q is CH₂, S or O. More preferably Q is O. Where Q is N-alkyl, suitably the alkyl is C₁ to C₅ alkyl. Where Q is CHalkyl or C(alkyl)₂, suitably the alkyl is C₁ to C₅ alkyl.

Preferably each of U and V is CH.

When a double bond exists between X and the ring atom to which Q is attached, X and Y are preferably both N.

When a double bond exists between X and the ring atom to which Q is attached, Z is preferably O.

When a double bond exists between X and the ring atom to which Q is attached, Q is preferably O.

When X and Y are N, Q and Z are independently preferably selected from O, S and NH, more preferably Q and Z are O.

Throughout the present specification:

alkyl includes cycloalkyl, alkyl substituted with cycloalkyl, alkyl containing within the alkyl chain 1, 2, 3 or 4 heteroatoms selected independently from O, S and N, substituted alkyl and branched alkyl;

alkenyl includes cycloalkenyl, alkyl substituted with cycloalkenyl, alkenyl containing within the alkenyl chain 1, 2, 3 or 4 heteroatoms selected independently from O, S and N for example tetrahydrofuran (THF), substituted alkenyl and branched alkenyl;

alkynyl includes cycloalkynyl, alkyl substituted with cycloalkynyl, alkynyl containing within the alkynyl chain 1, 2, 3 or 4 heteroatoms selected independently from O, S and N, substituted alkynyl and branched alkynyl; and

aryl includes monocyclic and bicyclic fused 5, 6 and 7 membered aromatic rings, aryl containing 1, 2, 3 or 4 heteroatoms selected independently from O, S and N, alkylaryl for example benzyl, and substituted aryl and substituted alkylaryl for example substituted benzyl.

The nature, position and number of any substituents and unsaturation present in any alkyl, alkenyl, alkynyl and aryl group may be varied.

Examples of suitable substituents on any of said alkyl, alkenyl, alkynyl and aryl, including alkylaryl, groups include OH, halogens, amino, CN, COOH, CO₂alkyl(C₁ to C₅), CONH₂, CONHalkyl(C₁ to C₅), O-alkyl(C₁ to C₅), SH, S-alkyl(C₁ to C₅) and NO₂, and aryl(5 to 10 ring atoms), and with respect to aryl and alkylaryl groups include alkyl (C₁ to C₅), alkenyl (C₂ to C₅) and alkynyl (C₂ to C₅), wherein any of said alkyl, alkenyl, alkynyl and aryl moieties are each optionally substituted. Substituents on the said alkyl, alkenyl and alkynyl moieties, which are preferably straight chain, can be selected from the group comprising OH, halogens, amino, CN, SH and NO₂, and is preferably a halogen, more preferably chlorine. Where the said alkyl, alkenyl or alkynyl moiety is C₂ to C₅, the substituent is preferably at the terminus position. Substituents on the said aryl moiety can be selected from the group comprising OH, halogens, amino, CN, NO₂, and C₁ to C₁₀ alkyl, which C₁ to C₁₀ alkyl moiety is optionally substituted with a member selected from the group comprising OH, halogens, amino, CN, SH, NO₂. The said aryl moiety can comprise aryl or heteroaryl groups. Any ring heteroatoms may vary in position or number. Suitably 1, 2, 3 or 4 heteroring atoms may be present, preferably selected, independently, from O, N and S. The said aryl moiety can comprise one, or two fused, 5, 6 or 7 membered rings.

Preferably R¹ is selected from C₃₋₂₀alkyl, C₃₋₂₀cycloalkyl, C₂₋₂₀alkenyl, C₃₋₂₀alkynyl, C₅₋₁₄ aryl and C₁₋₁₀alkylC₅₋₁₄aryl, more preferably C₃₋₁₄alkyl, C₃₋₁₄alkenyl, C₃₋₁₄alkynyl, more preferably C₆₋₁₄alkyl, C₆₋₁₄alkenyl, C₆₋₁₄alkynyl, even more preferably C₈₋₁₀alkyl, C₈₋₁₀ alkenyl and C₈₋₁₀alkynyl.

Preferably R¹ is C₄₋₁₄alkyl, C₄₋₁₄alkenyl or C₄₋₁₄alkynyl, more preferably C₄₋₁₂alkyl, C₄₋₁₂alkenyl or C₄₋₁₂alkynyl, even more preferably C₆₋₁₀alkyl, C₆₋₁₀alkenyl or C₆₋₁₀alkonyl, even more preferably C₈₋₁₀alkyl, C₈₋₁₀alkenyl or C₈₋₁₀alkynyl.

Where there is a single bond between X and the ring atom to which Q is attached, R¹ is preferably C₆₋₁₂alkyl, C₆₋₁₂alkenyl or C₆₋₁₂alkynyl.

Where there is a double bond between X and the ring atom to which Q is attached, R¹ is preferably C₄₋₁₂alkyl, C₄₋₁₂alkenyl or C₄₋₁₂alkynyl.

Preferably R¹ is an alkyl group. Preferably R¹ is a straight chain alkyl group. Preferably R¹ is an unsubstituted alkyl group. Preferably R¹ is a saturated alkyl group.

Preferably R¹ is a C₇ to C₁₃ alkyl group. More preferably R¹ is a C₈ to C₁₂ alkyl group, even more preferably a C₉ to C₁₁ alkyl group. Particularly preferred is R¹ being a C₉ or C₁₀ alkyl group.

Where R¹ is a straight chain alkyl group, a preferred position for substitution is the terminus position.

Suitably any substituent in R¹ is non-polar, more suitably any such substituent is additionally hydrophobic. Preferred substituents on R¹ include halogen and O-alkyl(C₁ to C₅). Particularly preferred is O-alkyl with C₄, optionally terminally substituted with a halogen, preferably chlorine.

When R¹ is a cycloalkyl group, it suitably comprises 5 to 12 ring carbon atoms arranged in one or two adjoining rings.

Preferably R¹ is selected from the group comprising nC₄H₉, nC₆H₁₃, nC₇H₁₅ and nC₁₀H₂₁. Preferably R¹ is nC₁₀H₂₁.

Preferably R⁴ and R⁸ are selected from C₁₋₁₂alkyl, C₂₋₁₂alkenyl, C₂₋₁₂alkynyl, C₃₋₁₂cycloalkyl, C₁₋₆alkyl substituted with C₃₋₁₀cycloalkyl, C₅₋₁₄aryl and C₁₋₅alkylC₅₋₁₄aryl.

Preferably R⁴ and R⁸ are selected from C₁₋₁₀alkyl C₂₋₁₀alkenyl, C₂₋₁₀alkynyl, C₁alkyl substituted with C₅₋₆cycloalkyl and C₁alkyl substituted with C₅₋₇aryl.

Even more preferably R⁴ and R⁸ are selected from C₁₋₆alkyl, C₂₋₄alkenyl, C₁alkyl substituted with C₅₋₆cycloalkyl and benzyl and substituted benzyl.

Preferably each of R⁴ and R⁸ are selected from the group comprising cycloC₅H₉, CH(Et)₂, nC₅H₁₁, 2-THF, CH₂cycloC₆H₁₁, 3-THF, cycloC₆H₁₁, C₃H₇, nC₄H₉, PhCH₂, TolCH₂, pMeOPhCH₂, CH₂cycloC₅H₉, Me and nC₃H₇.

Where a single bond exists between X and the ring atom to which Q is attached particularly preferred combinations of R¹ and R⁸ are, respectively, nC₇H₁₅ and cycloC₅H₉, nC₇H₁₅ and CH(Et)₂, nC₁₀H₂, and 3-THF, nC₁₀H₂₁ and cycloC₆H₁₁, nC₁₀H₂₁ and C₃H₇, nC₁₀H₂₁ and CH₂cycloC₅H₉, nC₆H₁₃ and Me, nC₆H₁₃ and nC₃H₇, and nC₆H₁₃ and PhCH₂. A particularly preferred combination is R¹ being nC₁₀H₂₁ and R⁸ being CH₂cycloC₅H₉.

Where a double bond exists between X and the ring atom to which Q is attached particularly preferred combinations of R¹ and R⁴ are, respectively, nC₄H₉ and cycloC₅H₉, nC₇H₁₅ and cycloC₅H₉, nC₇H₅ and CH(Et)₂, nC₇H₁₅ and nC₅H₁₁, nC₁₀H₂₁ and CH(Et)₂, nC₁₀H₂₁ and cycloC₆H₁₁, nC₁₀H₂₁ and nC₃H₇, nC₁₀H₂₁ and nC₄H₉, nC₁₀H₂₁ and PhCH₂, nC₁₀H₂, and CH₂cycloC₆H₁₁, nC₁₀H₂, and TolCH₂, nC₁₀H₂, and pMeOPhCH₂, nC₆H₁₃ and Me, nC₆H₁₃ and nC₄H₉, and nC₆H₁₃ and PhCH₂. Particularly preferred combinations are R¹ being nC₁₀H₂, with R⁴ being any of nC₃H₇, nC₄H₉, PhCH₂, CH₂cycloC₆H₁₁, tolCH₂, and pMeOPhCH₂.

Suitably R² is selected from the group comprising H, C₁ to C₁₀ alkyl, C₃ to C₁₀ cycloalkyl, C₁ to C₁₀ alkylamino, C₁ to C₁₀ dialkylamino, C₁ to C₁₀ alkyloxy, C₆ to C₁₀ aryloxy, C₁ to C₁₀ alkylthiol, C₆ to C₁₀ arylthiol and C₆ to C₁₀ aryl.

Suitably R³ is selected from the group comprising H, C₁ to C₁₀ alkyl, C₃ to C₁₀ cycloalkyl, C₁ to C₁₀ alkyloxy, C₆ to C₁₀ aryloxy and C₆ to C₁₀ aryl.

Preferably each of R² and R³ is a small alkyl i.e. a C₁ to C₂ alkyl group or H. More preferably each of R² and R³ is H.

Throughout the present specification “halogen” is taken to include any of F, Cl, Br and I.

Where not otherwise specified, alkyl is C₁₋₆alkyl, alkenyl is C₂₋₆alkenyl, alkynyl is C₂₋₆alkynyl, aryl is C₅₋₁₄aryl and alkylaryl is C₁₋₆alkylC₅₋₁₄aryl.

Where R¹, R⁴ or R⁸ is an aryl group, the group includes alkylaryl groups. Preferably R¹, R⁴ and R⁸ are C₅₋₁₄aryl groups or C₁₋₄alkylC₅₋₁₄ aryl groups. Particularly preferred groups are benzyl and substituted benzyl such as toluene (tol)CH₂, and pMeOPhCH₂. Preferred substituents include alkyl (C₁₋₆), alkoxy (C₁₋₆) and halogen (F, Cl, Br and I). The preferred substitution positions for phenyl and benzyl is para. Preferred aryl groups are C₆.

Where there is a single bond between X and the ring atom to which Q is attached:
when R¹ is alkyl, preferably R¹ is C₆₋₁₂alkyl;
when R¹ is alkynyl, preferably R¹ is C₈ or above alkynyl, more preferably C₈₋₂₀alkynyl, even more preferably C₈₋₁₄alkynyl; even more preferably C₈₋₁₂alkynyl; even more preferably C₈₋₁₀alkynyl;
when R¹ is aryl, preferably R¹ is a monocyclic or bicyclic fused 5, 6 or 7 membered ring, an aryl group containing 1, 2, 3 or 4 heteroatoms selected independently from O, S and N, alkylaryl for example benzyl, or substituted aryl or substituted alkylaryl for example substituted benzyl such as pMeOPhCH₂, more preferably a C₅₋₁₄aryl group or a C₁₋₄alkylC₅₋₁₄ aryl group, even more preferably a C₆ aryl group, the substitutents being as set out above;
when R¹ is alkyl containing within the alkyl chain 1, 2, 3 or 4 heteroatoms selected independently from O, S and N, preferably R¹ is not a thioether, even more preferably R¹ being a thioether is excluded from the scope of formula (I); and/or
when R⁸ is alkyl, R⁸ is not methyl when R¹ is n-butyl, Y is N, Z is O and V and U are CH.

The preferred options recited immediately above with respect to there being a single bond between X and the ring atom to which Q is attached do not necessarily extend to those aspects of the present invention recited below directed, respectively, to: a compound according to the present invention for use in a method of treatment, suitably in the prophylaxis or treatment of a viral infection, preferably a cytomegalovirus infection; a method of prophylaxis or treatment of a viral infection, preferably a cytomegalovirus infection; and use of a compound of the present invention in the manufacture of a medicament for use in the prophylaxis or treatment of a viral infection, particularly an infection with cytomegalovirus.

According to a further aspect of the present invention there is provided a method for preparing compounds having Formula I above wherein a 5-halo nucleoside analogue is contacted with a terminal alkyne in the presence of a catalyst. Alternatively 5-alkynyl nucleoside can be cyclised in the presence of a catalyst. Suitably the catalyst is a copper catalyst.

Compounds of the present invention may be prepared by a number of methods, which may for example involve a reaction scheme such as:

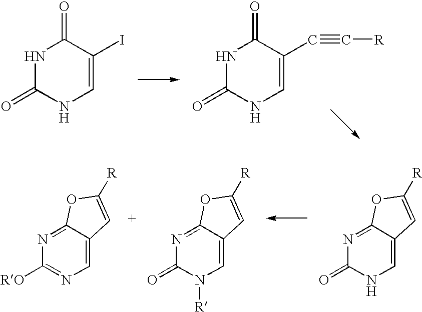

Thus, terminal acetylenes are coupled to 5-iodouracil under Pd catalysed conditions to give intermediate 5-alkynyl compounds that may either be isolated or used in situ. These are cyclised under Cu catalysis to give bicyclic furano pyrimidines that are key synthons. These are alkylated to give mixtures of O and N alkyl products that can be readily separated.

The method of separation may include chromatography, precipitation, and crystallisation. The ratios of these products will vary, and need not be 1:1.

Compounds embodying the present invention can show anti-viral activity. In particular, it has surprisingly been found that compounds embodying the present invention can show antiviral activity against for example cytomegalovirus.

According to a further aspect of the present invention there is provided a compound according to the present invention for use in a method of treatment, suitably in the prophylaxis or treatment of a viral infection, preferably a cytomegalovirus viral infection.

According to a further aspect of the present invention there is provided a method of prophylaxis or treatment of viral infection, preferably a cytomegalovirus viral infection, comprising administration to a patient in need of such treatment an effective dose of a compound according to the present invention.

According to a further aspect of the present invention there is provided use of a compound of the present invention in the manufacture of a medicament for use in the prophylaxis or treatment of a viral infection, particularly an infection with cytomegalovirus.

According to a further aspect of the present invention there is provided a pharmaceutical composition comprising a compound of the present invention in combination with a pharmaceutically acceptable excipient.

According to a further aspect of the present invention there is provided a method of preparing a pharmaceutical composition comprising the step of combining a compound of the present invention with a pharmaceutically acceptable excipient.

The compounds embodying the present invention present a number of advantages over existing agents for HCMV:
1. A novel non-nucleoside structure and possibly novel mechanism of action.
2. Antiviral activity at non-cytotoxic concentrations.
3. A lack of cross resistance with existing nucleoside drugs.
4. Useful physiochemical properties such as high lipophilicity. Lead structures have calculated logp (ClogP) values of Ca. 4-6.

The high lipophilicity of the present compounds may lead to improved in vivo dosing, tissue distribution and pharmacokinetics. In a preliminary rodent trial a compound with structure 5 with R¹═C₇H₁₅ and R⁴=cyclopentyl displayed significant bioavailability and half life following i.p. dosing. Moreover at a dose as high as 50 mg/kg/day for 10 days no visible in vivo toxicity was noted, indicating a promising toxicology profile. Histology also revealed no detectable toxicity against brain, thymus, liver, lungs, kidney, breast, testi, ovum and spleen tissue.

The compounds embodying the present invention can be sufficiently lipophilic to warrant their formulation and use as non-p.o dosage forms including topical, transdermal and ocular formulations. The latter may be of particular value versus HCMV retinitis, common in persons co-infected with HIV. The agents would therein have significant dosing, tissue localisation and toxicology advantage over current agents.

The lack of chirality in structures embodying the present invention distinguishes them from typical nucleoside antivirals with possible costs of goods and ease of synthesis advantage.

The medicaments employed in the present invention can be administered by oral (p.o.) or parenteral (i.p.) routes, including intravenous, intramuscular, intraperitoneal, subcutaneous, transdermal, airway (aerosol), rectal, vaginal and topical (including buccal and sublingual) administration.

For oral administration, the compound of the invention will generally be provided in the form of tablets or capsules, as a powder or granules, or as an aqueous solution or suspension.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

In general a suitable dose will be in the range of 0.1 to 300 mg per kilogram body weight of the recipient per day, preferably in the range of 1 to 25 mg per kilogram body weight per day and most preferably in the range 5 to 10 mg per kilogram body weight per day. The desired dose is preferably presented as two, three, four, five or six or more sub-doses administered at appropriate intervals throughout the day. These sub-doses may be administered in unit dosage forms, for example, containing 10 to 1500 mg, preferably 20 to 1000 mg, and most preferably 50 to 700 mg of active ingredient per unit dosage form.

Embodiments of the present invention will now be described by way of example only.

All reagents and solvents were obtained commercially and use without further purification, unless otherwise stated. Reaction progress was monitored by thin-layer chromatography (TLC) on DC-Alufolien 60F₂₅₄ 0.2 mm plates. Compounds were visualised by UV fluorescence (wavelength 365 nm). The reaction mixtures were evaporated in a vacuum rotary evaporator (Büchi Rotavapor R-114) using the vacuum of a diaphragm pump. This process is referred to below as “evaporated/removed/distilled in vacuo” or “under reduced pressure”. Flash column chromatography refers to the technique described by Still (Still et al J. Org. Chem. 1978, 43 (14), 2923-2925). The height of the silica gel 60 (220-440 mesh) in all cases was 15 cm. All air and moisture sensitive reactions were carried out under a nitrogen atmosphere in oven-dried glassware. Reaction mixture temperatures were measured externally.

¹H and ¹³C NMR spectra were recorded on a Bruker Avance DPX300 spectrometer at 300 MHz and 75.5 MHz respectively, with the corresponding deuterated solvents noted. The chemical shifts are reported in parts per million relative to the residual non-deuterated solvent peak (δ_{H} CHCl₃ 7.27; δ_{H} [D₅]DMSO 2.50; and δ_{c} CHCl₃ 77.0 and δ_{c} [D₅]DMSO 39.5 central peak). J values are given in Hz. The DEPT and NOE techniques were used to assign different carbon atoms. Chemical shifts are reported: value (splitting pattern, number of protons, coupling constant (where applicable), and assignment). Splitting pattern is designated as follows: s, singlet; app d, apparent doublet; d, doublet; dd, double doublet; t, triplet; q, quartet; quin, quintet; sex, sextet; sept, septet; m, multiplet; and br, broad. Elemental analyses were carried out in the Microanalytical Laboratories of the School of Pharmacy, University of London.

### 6-Heptyl-3H-furo[2,3-d]pyrimidin-2-one (137)

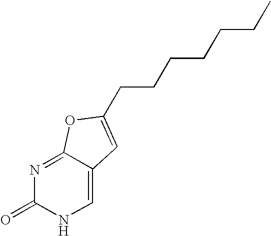

To a stirred solution of 5-Iodo-uracil (3.00 g, 12.60 mmol) in dry dimethylformamide (30 ml) at room temperature and under a nitrogen atmosphere, 1-hexyne (4.20 ml, 37.80 mmol), tetrakis(triphenylphosphine)palladium(0) (728 mg, 0.63 mmol), copper (I) iodide (240 mg, 1.26 mmol), and diisopropylethylamine (4.4 ml, 25.20 mmol), were added. The reaction mixture was stirred at room temperature for 19 hours, after which time TLC (chloroform/methanol 95:5) showed complete conversion of the starting material. Copper(I) iodide (240 mg, 1.26 mmol), triethylamine (20 ml) was added to the mixture which was subsequently refluxed for 8 hours. The reaction mixture was then concentrated in vacuo, and the product was purified by trituration with methanol, (1.20 g, 41%).

¹H-nmr (d₆-DMSO; 300 MHz): 11.97 (1H, bs, NH), 8.15 (1H, s, H-4) 6.37 (1H, s, H-5), 2.60 (2H, t, J=7.3 Hz, α-CH₂), 1.62 (2H, m, CH₂), 1.28 (8H, m, 4×CH₂), 0.86 (3H, t, J=7.2 Hz, CH₃).

¹³C-nmr unavailable due to solubility problems.

### 6-Butyl-3-cyclopentyl-3H-furo[2,3-d]pyrimidin-2-one (138) [Cf2158]

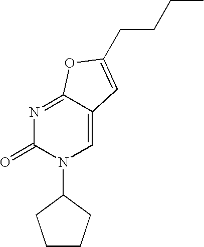

To a suspension of 6-Butyl-3H-furo[2,3-d]pyrimidin-2-one (136) (350 mg, 1.82 mmol) in dry DMF (20 ml) under an atmosphere of nitrogen, potassium carbonate (502 mg, 3.64 mol) and cyclopentyl bromide (0,39 ml, 3.64 mmol) were added. The reaction mixture as stirred at 80° C. for one hour. The solvent was evaporated in vacuo and the residue was dissolved in dichloromethane and extracted with a saturated solution of sodium chloride. The extracts were collected, dried on magnesium sulphate and evaporated to dryness. The crude product was purified by silica column chromatography, using chloroform as eluent, followed by a mixture of chloroform/methanol (97:3). The appropriate fractions were combined and the solvent was removed in vacuo to yield the product which was flirter purified by trituration with diethyl ether, yielding the pure product (47 mg, 10%) as a white solid. Mp: 130-131° C.

¹H-nmr (CDCl₃; 300 MHz): 7.84 (1H, s, H-4) 6.13 (1H, s, H-5), 5.29 (1H, m, CH), 2.69 (2H, t, J=7.2 Hz, α-CH₂), 2.33 (2H, m, cyclopentyl-CH₂), 2.01-1.67 (8H, m, cyclopentyl+CH₂), 1.45 (2H, m, CH₂), 0.99 (3H, t, J=7.3 Hz, CH₃).

¹³C-nmr (CDCl₃; 75 MHz): 14.1 (CH₃), 22.5, 28.4, 29.3 (3×CH₂), 24.5, 32.8 (cyclopentyl-CH₂), 59.6 (CH), 98.9 (C-5) 108.2 (C-4a), 135.6 (C-4), 156.2 (C-6), 160.3 (C-2), 171.3 (C-7a).

MS (ES+) m/e 283 (MNa⁺, 100%)

Accurate mass: C₁₅H₂₀N₂O₂Na requires 283.1422; found 283.1414.

### 6-Butyl-2-cyclopentyloxy-furo[2,3-d]pyrimidine (139) [Cf2159]

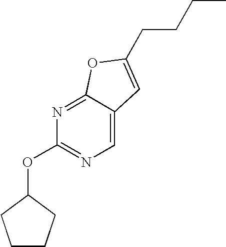

Also isolated from the above reaction as a white solid (270 mg, 57%). Mp: 69-71° C.

¹H-nmr (CDCl₃; 300 MHz): 8.61 (1H, s, H-4) 6.42 (1H, s, H-5), 5.48 (1H, m, CH), 2.78 (2H, t, J=7.2 Hz, α-CH₂), 2.06-1.67 (10H, m, cyclopentyl+β-CH₂), 1.46 (2H, m, χ-CH₂), 0.99 (3H, t, J=7.3 Hz, CH₃).

¹³C-nmr (CDCl₃; 75 MHz): 14.2 (CH₃), 22.6, 28.4, 29.7 (3×CH₂), 24.2, 33.2 (cyclopentyl-CH₂), 80.4 (CH), 99.5 (C-5) 113.9 (C-4a), 150.9 (C-4), 158.9 (C-6), 162.6 (C-2), 168.8 (C-7a).

MS (ES+) m/e 283 (MNa⁺, 100%)

Accurate mass: C₁₅H₂₀N₂O₂Na requires 283.1422; found 283.1428.

### 6-Heptyl-3-cyclopentyl-3H-furo[2,3-d]pyridin-2-one (140) [Cf2160]

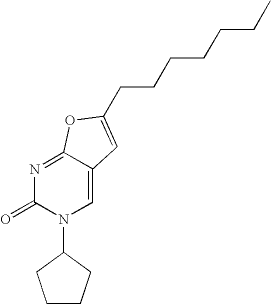

This was synthesised as described for 138 above, using 350 mg of 137 (1.49 mmol) and 0.32 ml of cyclopentyl bromide (2.98 mmol). The product was collected as a white solid (88 mg, 20%). Mp: 142-143° C.

IR (KBr): 2930.6 (aliphatic), 1677.8 (CO amide).

¹H-nmr (CDCl₃; 300 MHz): 7.80 (1H, s, H-4) 6.09 (1H, s, H-5), 5.25 (1H, m, CH), 2.64 (2H, t, J=7.4 Hz, α-CH₂), 2.25 (2H, m, cyclopentyl-CH₂), 1.90-1.67 (8H, m, 4×CH₂), 1.34 (8H, m, 4×CH₂), 0.88 (3H, t, J=6.7 Hz, CH₃). ¹³C-nmr (CDCl₃; 75 MHz): 14.5 (CH₃), 23.0, 27.2, 27.9, 29.3, 29.7, 32.8 (6×CH₂), 24.5, 33.1 (cyclopentyl-CH₂), 59.7 (CH), 98.9 (C-5) 108.2 (C-4a), 135.7 (C4), 156.2 (C-6), 160.3 (C-2), 171.6 (C-7a).

MS (ES+) m/e 325 (MNa⁺, 100%)

Accurate mass: C₁₈H₂₆N₂O₂Na requires 325.1892; found 325.1883

### 6-Heptyl-2-cyclopentyloxy-furo[2,3-d]pyrimidine (141) [Cf2161]

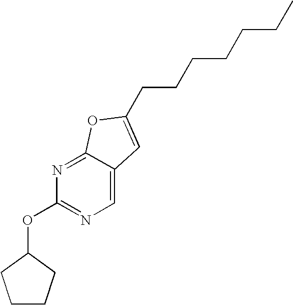

Also isolated from the above reaction as a white solid (230 mg, 51%). Mp: 65-67° C.

IR (KBr): 2954.1 (aliphatic), 1619.6 (C═N).

¹H-nmr (CDCl₃; 300 MHz): 8.60 (1H, s, H-4) 6.36 (1H, s, H-5), 5.48 (1H, m, CH), 2.77 (2H, t, J=7.3 Hz, α-CH₂), 2.08-1.63 (10H, m, cyclopentyl+β-CH₂), 1.42-1.27 (8H, m, 4×CH₂), 0.91 (3H, t, J=7.2 Hz, CH₃).

¹³C-nmr (CDCl₃; 75 MHz): 14.5 (CH₃), 23.0, 27.6, 28.8, 29.4, 29.4, 32.1 (6×CH₂), 24.2, 33.2 (cyclopentyl-CH₂), 80.4 (CH), 99.5 (C-5) 113.9 (C-4a), 150.9 (C4), 158.9 (C-6), 162.6(C-2), 168.8 (C-7a).

MS (ES+) m/e 325 (MNa⁺, 100%)

Accurate mass: C₁₈H₂₆N₂O₂Na requires 325.1892; found 325.1880

### 6-Butyl-3-(1-ethyl-propyl)-3H-furo[2,3-d]pyrimidin-2-one (142) [Cf2194]

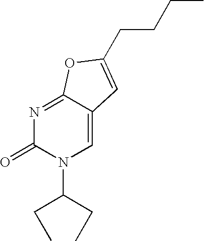

This was synthesised as described for 138 above, using 300 mg of 136 (1.56 mmol) and 0.40 ml of 3-bromopentane (3.12 mmol). The product was collected as a white solid (118 mg, 29%).

IR (KBr): 2958.1 (aliphatic), 1671.9 (CO amide).

¹H-nmr (CDCl₃; 300 MHz): 7.72 (1H, s, H-4) 6.14 (1H, s, H-5), 4.94 (1H, m, CH), 2.68 (2H, t, J=7.4 Hz, α-CH₂), 1.93-1.66 (6H, m, 3×CH₂), 1.43 (2H, m, CH₂), 1.00-0.88 (9H, m, 3×CH₃).

¹³C-nmr (CDCl₃; 75 MHz): 10.7, 14.1 (3×CH₃), 22.5, 27.9, 28.4, 29.3 (5×CH₂), 61.3 (CH), 98.9 (C-5) 108.2 (C-4a), 135.4 (C-4), 156.7 (C-6), 160.3 (C-2), 171.4 (C-7a).

MS (ES+) m/e 285 (MNa⁺, 100%)

Accurate mass: C₁₅H₂₂N₂O₂Na requires 285.1579; found 285.1586

Anal. Calcd for C₁₅H₂₂N₂O₂: C, 68.67%; H, 8.45%; N, 10.68%. Found: C, 68.38%; H, 8.62%; N, 10.89%

### 6-Butyl-2-(1-ethyl-propoxy)-furo[2,3-d]pyrimidine (143) [Cf2193]

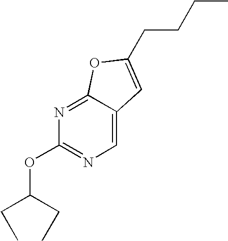

Also isolated from the above reaction as a white solid (171 mg, 42%).

IR (KBr): 2938.4 (aliphatic), 1620.0 (C═N).

¹H-nmr (CDCl₃; 300 MHz): 8.60 (1H, s, H-4) 6.35 (1H, s, H-5), 5.10 (1H, m, CH), 2.77 (2H, t, J=7.4 Hz, α-CH₂), 1.91-1.70 (6H, m, 3×CH₂), 1.43 (2H, m, CH₂), 1.00-0.90 (9H, m, 3×CH₃).

¹³C-nmr (CDCl₃; 75 MHz): 10.0, 14.1 (3×CH₃), 22.6, 26.5, 28.4, 29.7 (5×CH₂), 80.0 (CH), 99.5 (C-5) 113.9 (C-4a), 150.9 (C-4), 158.9 (C-6), 162.9 (C-2), 168.8 (C-7a).

MS (ES+) m/e 285 (MNa⁺, 100%)

Accurate mass: C₁₅H₂₂N₂O₂Na requires 285.1579; found 285.1575

Anal. Calcd for C₁₅H₂₂N₂O₂: C, 68.67%; H, 8.45%; N, 10.68%. Found: C, 66.97%; H, 8.58%; N, 10.78%

### 6-Heptyl-3-(1-ethyl-propyl)-3H-furo[2,3-d]pyrimidin-2-one (144) [Cf2190]

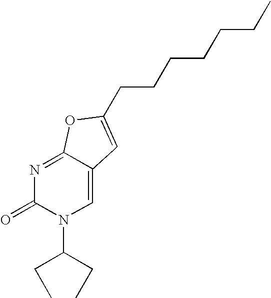

This was synthesised as described for 138 above, using 350 mg of 137 (1.50 mmol) and 0.40 ml of 3-bromopentane (3.00 mmol). The product was collected as a white solid (108 g, 28%). Mp: 128-130° C.

¹H-nmr (CDCl₃; 300 MHz): 7.71 (1H, s, H-4) 6.14 (1H, s, H-5), 4.94 (1H, m, CH), 2.68 (2H, t, J=7.4 Hz, α-CH₂), 1.96-1.67 (6H, m, 3×CH₂), 1.43-1.32 (8H, m, 4×CH₂), 0.98-0.89 (9H, m, 3×CH₃).

¹³C-nmr (CDCl₃; 75 MHz): 10.7, 14.5 (3×CH₃), 23.0, 27.2, 27.9, 28.7, 29.3 29.4, 32.1 (7×CH₂), 61.3 (CH), 98.9 (C-5) 108.2 (C-4a), 135.4 (C4), 156.7 (C-6), 160.3 (C-2), 171.4 (C-7a).

MS (ES+) m/e 327 (MNa⁺, 100%), 305 (MH⁺) (50%)

Accurate mass: C₁₈H₂₈N₂O₂Na requires 327.2048; found 327.2038

### 6-Heptyl-2-(1-ethyl-propoxy)-furo[2,3-d]pyrimidine (145) [Cf2189]

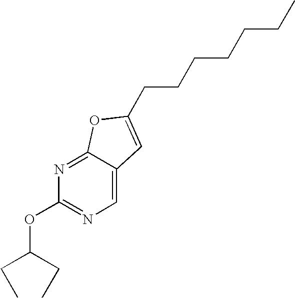

Also isolated from the above reaction as a white solid (272 mg, 70%). Mp: 70-71° C.

¹H-nmr (CDCl₃; 300 MHz): 8.48 (1H, s, H-4) 6.24 (1H, s, H-5), 5.01 (1H, m, CH), 2.65 (2H, t, J=7.3 Hz, α-CH₂), 1.72-1.60 (6H, m, 3×CH₂), 1.60-1.20 (8H, m, 4×CH₂), 0.91-0.77 (9H, m, 3×CH₃).

¹³C-nmr (CDCl₃; 75 MHz): 9.9, 14.4 (3×CH₃), 23.0, 26.4, 27.6, 28.7, 29.3, 29.4, 32.0 (7×CH₂), 80.0 (CH), 99.5 (C-5) 113.9 (C-4a), 150.9 (C-4), 158.8 (C-6), 162.9 (C-2), 168.8 (C-7a).

MS (ES+) m/e 327 (MNa⁺, 100%)

Accurate mass: C₁₈H₂₈N₂O₂Na requires 327.2048; found 327.2053

### 6-Butyl-3-pentyl-3H-furo[2,3-d]pyrimidin-2-one (146) [Cf2195]

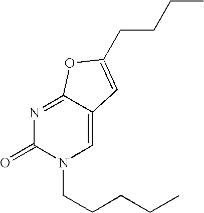

This was synthesised as described for 138 above, using 250 mg of 136 (1.30 mmol) and 515 mg of 1-Iodopentane (2.60 mmol). The product was collected as a white solid (133 mg, 40%). Mp: 139-141° C.

¹H-nmr (CDCl₃; 300 MHz): 7.77 (1H, s, H-4) 6.07 (1H, s, H-5), 3.96 (2H, t, J=7.4 Hz, N—CH₂), 2.61 (2H, t, J=7.4 Hz, α-CH₂), 1.94-1.58 (4H, m, 3×CH₂), 1.43-1.24 (6H, m, 3×CH₂), 0.93-0.84 (6H, m, 2×CH₃).

¹³C-nmr (CDCl₃; 75 MHz): 14.1, 14.3 (2×CH₃), 22.5, 22.7, 28.4, 29.0, 29.2, 29.3 (6×CH₂), 52.6 (N—CH₂), 98.8 (C-5) 108.1 (C-4a), 139.1 (C4), 155.8 (C-6), 160.2 (C-2), 172.3 (C-7a).

MS (ES+) m/e 285 (MNa⁺, 100%)

Accurate mass: C₁₅H₂₂N₂O₂Na requires 285.1579; found 285.1568

### 6-Butyl-2-pentyloxy-furo[2,3-d]pyridine (147) [Cf 2327]

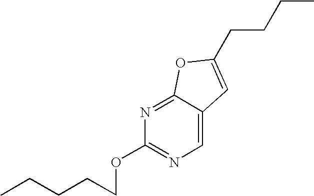

Also isolated from the above reaction as a white solid (62 mg, 20%). Mp: 51-52° C.

¹H-nmr (CDCl₃; 300 MHz): 8.49 (1H, s, H-4) 6.25 (1H, s, H-5), 4.32 (2H, t, J=6.6 Hz, O—CH₂), 2.64 (2H, t, J=7.3 Hz, α-CH₂), 1.85-1.66 (4H, m, 2×CH₂), 1.43 (6H, m, 3×CH₂), 0.92-0.73 (6H, m, 2×CH₃).

¹³C-nmr (CDCl₃; 75 MHz): 14.1, 14.4 (2×CH₃), 22.5, 22.8, 28.4, 28.5, 28.9, 29.6 (7×CH₂), 68.3 (O—CH₂), 99.5 (C-5) 114.1 (C-4a), 150.9 (C-4), 159.0 (C-6), 162.8 (C-2), 168.8(C-7a).

MS (ES+) m/e 285 (MNa⁺, 100%)

Accurate mass: C₁₅H₂₂N₂O₂Na requires 285.1579; found 285.1584

### 6-Heptyl-3-pentyl-3H-furo[2,3-d]pyrimidin-2-one (148) [Cf2192]

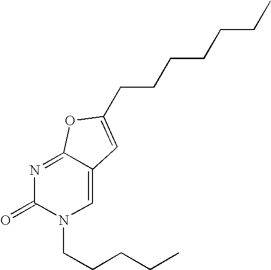

This was synthesised as described for 138 above, using 350 mg of 137 (1.50 mmol) and 594 mg of 1-Iodopentane (3.00 mmol). The product was collected as a white solid (207 mg, 45%). Mp: 161-162° C.

IR (KBr): 2922.1 (aliphatic), 1678.3 (CO amide).

¹H-nmr (CDCl₃; 300 MHz): 7.87 (1H, s, H-4) 6.18 (1H, s, H-5), 4.07 (2H, t, J=7.4 Hz, N—CH₂), 2.71 (2H, t, J=7.3 Hz, α-CH₂), 1.93-1.71 (4H, m, 2×CH₂), 1.42 (12H, m, 6×CH₂), 0.98 (6H, m, 2×CH₃).

¹³C-nmr (CDCl₃; 75 MHz): 14.3, 14.9 (2×CH₃), 22.7, 23.0, 27.2, 28.7, 29.1, 29.3, 29.3 29.4 32.1 (9×CH₂), 52.6 (N—CH₂), 98.8 (C-5) 108.1 (C-4a), 139.1 (C4), 155.8 (C-6), 160.3 (C-2), 172.3 (C-7a).

MS (ES+) m/e 327 (MNa⁺, 100%)

Accurate mass: C₁₈H₂₈N₂O₂Na requires 327.2048; found 327.2042

### 6-Heptyl-2-pentyloxy-furo[2,3-d]pyrimidine (149) [Cf2191]

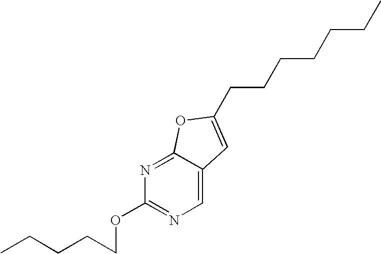

Also isolated from the above reaction as a white solid (141 mg, 31%). Mp: 48-49° C.

IR (KBr): 2933.0 (aliphatic), 1618.0 (C═N).

¹H-nmr (CDCl₃; 300 MHz): 8.50 (1H, s, H-4) 6.25 (1H, s, H-5), 4.30 (2H, t, J=6.7 Hz, O—CH₂), 2.65 (2H, t, J=7.4 Hz, α-CH₂), 1.80-1.60 (4H, m, 2×CH₂), 1.44-1.19 (12H, m, 6×CH₂), 0.86-0.77 (6H, m, 2×CH₃).

¹³C-nmr (CDCl₃; 75 MHz): 14.4 (2×CH₃), 22.8, 23.0, 27.6, 28.5, 28.7, 28.9, 29.3, 29.4, 32.0 (9×CH₂), 68.3 (O—CH₂), 99.5 (C-5) 114.1 (C-4a), 150.8 (C-4), 159.0 (C-6), 162.8 (C-2), 172.3 (C-7a).

MS (ES+) m/e 327 (MNa⁺, 100%)

Accurate mass: C₁₈H₂₈N₂O₂Na requires 327.2048; found 327.2050

### 6-Heptyl-3-(tetrahydro-furan-2-yl)-3H-furo[2,3-d]pyrimidin-2-one (154) [Cf2196]

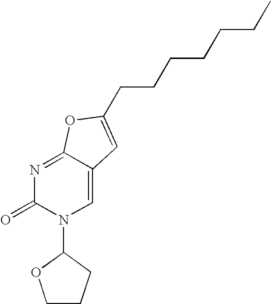

To a suspension of 6-heptyl-3H-furo[2,3-d]pyrimidin-2-one (137) (288 mg, 1.23 mmol) in dry DMF (10 ml) 2-tert-Butoxytetrahydrofuran (709 mg, 4.92 mmol) was added. The reaction mixture was stirred at 150° C. for 10 hours. The solvent was evaporated in vacuo and the residue was dissolved in dichloromethane and purified by silica column chromatography, using chloroform as eluent, followed by a mixture of chloroform/methanol (98:2). The appropriate fractions were combined and the solvent was removed in vacuo to yield the product, which was ether purified by trituration with diethyl ether, yielding the pure product (150 mg, 40%) as a white solid.

IR (KBr): 2927.1 (aliphatic), 1671.9 (CO amide), 1084.0 (C—O).

¹H-nmr (CDCl₃; 300 MHz): 7.93 (1H, s, H-4) 6.09 (2H, m, H-5 and H-1′), 4.26 and 4.04 (2H, m, H-5′), 2.60 (2H, t, J=7.4 Hz, α-CH₂), 2.56 (2H, m, H-2′ₐ), 2.18 and 2.00 (2H, m, H-3) 1.99 (2H, m, H-2′_{b}), 1.59 (2H, m, CH₂), 1.30-1.23 (8H, m, 4×CH₂), 0.83 (3H, t, J=6.7 Hz, CH₃).

¹³C-nmr (CDCl₃; 75 MHz): 14.5 (CH₃), 23.0, 23.7, 27.2, 28.7, 29.3, 29.4, 32.1, 33.8 (8×CH₂), 71.1 (C-5′), 90.2 (C-1′), 99.1 (C-5), 107.6 (C-4a), 134.2 (C-4), 155.2 (C-6), 160.2 (C-2), 171.3 (C-7a).

MS (ES+) m/e 327 (MNa⁺, 100%)

Accurate mass: C₁₇H₂₄N₂O₃Na requires 327.1685; found 327.1678

Anal. Calcd for C₁₇H₂₄N₂O₃: C, 67.08%; H, 7.95%; N, 9.20%. Found: C, 67.01%; H, 8.14%; N, 9.26%

### 6-Decyl-2,3-dihydrofuro[2,3-d]pyrimidin-2-one 26

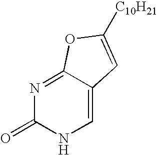

To a dry DMF (50 mL) solution of 5-iodouracil 23 (5.00 g, 21 mmol), tetrakis(triphenylphosphine)palladium(0) (1.00 g, 0.87 mmol, 0.04 equiv.) and copper iodide (0.80 g, 4.2 mmol, 0.2 equiv.) under a nitrogen atmosphere was added dry DIPEA (7.3 mL, 5.42 g, 42 mmol, 2 equiv.) and 1-dodecyne 24 (13.5 mL, 10.48 g, 63 mmol, 3 equiv.) via syringe with stirring. The initially opaque yellow solution proceeded to change colour on stirring at room temperature to a clear dark yellow solution, and eventually an opaque dark green suspension formed after a couple of hours. The suspension was allowed to react at RT with stirring for 18 h. TLC analysis of the resulting mixture indicated that most of the starting material had reacted, and the presence of a blue fluorescent spot was clearly observed. Dry triethylamine (25 mL) and a further addition of copper iodide (0.80 g) was then made to the suspension, and the resultant reaction mixture heated to 80° C. for 6 h with stirring under N₂. The suspension was allowed to cool to RT overnight with stirring. The resultant precipitate was collected by suction filtration, and washed consecutively with methanol and DCM. The collected solid was triturated in hot methanol to yield the title compound 26 as a white insoluble solid of weight 3.79 g (65% from 23).

### 6-Decyl-2-propoxy-furo[2,3-d]pyrimidine Cf2303

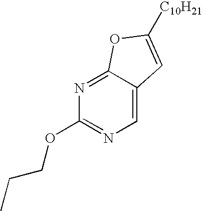

26 (0.30 g, 1.086 mmol), potassium carbonate (0.30 g, 2.17 mmol, 2 equiv) and 1-iodopropane (30, 0.22 mL, 2.17 mmol, 2 equiv.) were suspended in dry DMF (5 mL) under N₂, and the reaction mixture heated to 100° C. with stirring overnight. The solvent was then removed in vacuo at 80° C., and the crude mixture purified by flash chromatography in a 0-5% methanol/DCM eluent gradient to yield 31 (102 mg, 29%), the title compound, as a white solid. ¹H NMR (CDCl₃) δ 8.48 (s, 1H, 4-H), 6.49 (s, 1H, 5-H), 4.44 (t, J=6.7 Hz, 2H, O—CH₂—), 2.81 (t, J=7.6 Hz, 2H, 1′-CH₂), 1.95 (app sex, J=7.1 Hz, 2H, CH₂), 1.82 (m, J=6.6 Hz, 2H, CH₂), 1.43 (m, 14H, CH₂), 1.15 (t, J=7.4 Hz, 3H, O—CH₂CH₃), 0.97 (t, J=7.0 Hz, 3H, —CH₂CH₃); ¹³C NMR (CDCl₃) δ 168.9 (7a-C), 162.9 (2-C), 159.1 (6-C), 150.9 (4-CH), 114.2 (4a-C), 99.5 (5-CH), 69.9 (O—CH₂), 32.3 (1′-CH₂), 30.0 (CH₂), 29.9 (CH₂), 29.7 (CH₂), 29.6 (CH₂), 29.5 (CH₂), 28.8 (CH₂), 27.6 (CH₂), 23.1 (CH₂), 22.6 (CH₂), 14.5 (O—CH₂CH₃), 10.9 (—CH₂CH₃).

### 6-Decyl-3-propyl-3H-furo[2,3-d]pyrimidin-2-one Cf2304

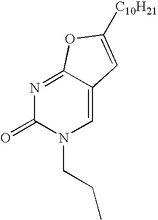

Also isolated from the mix was 191 mg of the title compound 32 (55% yield) as a white solid ¹H NMR (CDCl₃) δ 7.74(s, 1H, 4-H), 6.13(s, 1H, 5-H), 4.01 (t, J=7.3 Hz, 2H, N—CH₂—), 2.70 (t, J=7.7 Hz, 2H, 1′-CH₂), 1.89 (app sex, J=7.4 Hz, 2H, CH₂), 1.89 (m, J=7.4 Hz, 2H, CH₂), 1.70 (m, J=7.4 Hz, 2H, CH₂), 1.38 (m, 14H, CH₂), 1.04 (t, J=7.4 Hz, 3H, N—CH₂CH₃), 0.96 (t, J=7.0 Hz, 3H, —CH₂CH₃); ¹³C NMR (CDCl₃) δ 169.9 (7a-C), 160.4 (2-C), 156.1 (6-C), 138.9 (4-CH), 108.6 (4a-C), 98.6 (5-CH), 54.2 (N—CH₂), 32.3 (1′-CH₂), 30.0 (CH₂), 29.9 (CH₂), 29.7 (CH₂), 29.6 (CH₂), 29.5 (CH₂), 28.7 (CH₂), 27.2 (CH₂), 23.1 (CH₂), 22.8 (CH₂), 14.5 (O—CH₂CH₃), 11.5 (—CH₂CH₃).

### 2-Butoxy-6decyl-furo[2,3-a]pyrimidine Cf2305

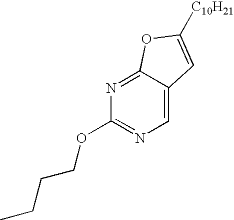

26 (0.30 g, 1.086 mmol), potassium carbonate (0.30 g, 2.17 mmol, 2 equiv) and 1-iodobutane 33 (0.25 mL, 2.17 mmol, 2 equiv.) were suspended in dry DMF (5 mL) under N₂, and the reaction mixture heated to 100° C. with stirring overnight. The solvent was then removed in vacuo at 80° C., and the crude mixture purified by flash chromatography in a 0-5% methanol/DCM eluent gradient to yield 34 (114 mg, 32%) as white solid.

¹H NMR (CDCl₃) δ8.61 (s, 1H, 4-H), 6.36 (s, 1H, 5-H), 4.36 (t, J=6.7 Hz, 2H, O—CH₂—), 2.75 (t, J=7.6 Hz, 2H, 1′-CH₂), 1.90-1.74 (m, 4H, CH₂), 1.54 (m, 2H, CH₂), 1.29 (m, 14H, CH₂), 1.00 (t, J=6.8 Hz, 3H, O—CH₂CH₃), 0.91 (t, J=7.0 Hz, 3H, —CH₂CH₃); ¹³C NMR (CDCl₃) δ 168.9 (7a-C), 162.9 (2-C), 159.1 (6-C), 150.9 (4-CH), 113.9 (4a-C), 99.5 (5-CH), 68.1 (O—CH₂), 32.3 (1′-CH₂), 31.3 (CH₂), 30.0 (CH₂), 29.9 (CH₂), 29.7 (CH₂), 29.6 (CH₂), 29.5 (CH₂), 28.8 (CH₂), 27.6 (CH₂), 23.1 (CH₂), 19.6 (CH₂), 14.5 (O—CH₂CH₃), 14.2 (—CH₂CH₃).

### 3-Butyl-6-decyl-3H-furo[2,3-d]pyrimidin-2-one Cf2306

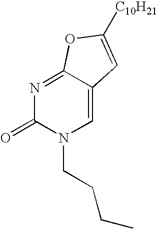

Also isolated from the mixture was the title compound 35 (205 mg, 57% yield) as a white solid.

¹H NMR (CDCl₃) δ 7.76 (s, 1H, 4-H), 6.04 (s, 1H, 5-H), 3.93 (t, J=7.4 Hz, 2H, N—CH₂—), 2.56 (t, J=7.4 Hz, 2H, 1′-CH₂), 1.71 (m, 2H, CH₂), 1.60 (m, 2H, CH₂), 1.36-1.18 (m, 16H, CH₂), 0.88 (t, J=7.2 Hz, 3H, N—CH₂—CH₃), 0.80 (t, J=6.5 Hz, 3H, —CH₂CH₃); ¹³C NMR (CDCl₃) δ 172.3 (7a-C), 160.3 (2-C), 155.9 (6-C), 139.2 (4-CH), 108.2 (4a-C), 98.8 (5-CH), 52.4 (N—CH₂—), 32.3 (1′-CH₂), 31.6 (CH₂), 30.0 (CH₂), 29.9 (CH₂), 29.7 (CH₂), 29.5 (CH₂), 29.4 (CH₂), 28.7 (CH₂), 27.2 (CH₂), 23.1 (CH₂), 20.2 (CH₂), 14.5 (O—CH₂CH₃), 14.1 (—CH₂CH₃).

### 6-Decyl-2-pentyloxy-2,3-dihydrofuro[2,3-d]pyrimidine Cf2247

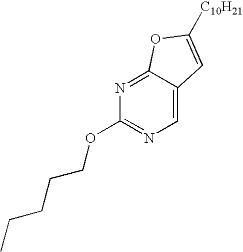

6-Decyl-2,3-dihydrofuro[2,3-d]pyrimidin-2-one 26 (200 mg, 0.72 mmol), potassium carbonate (199 mg, 1.44 mmol, 2 equiv.) and 1-iodopentane 36 (0.2 mL, 2 equiv.) were suspended in dry DMF (8 mL) under N₂, and the suspension heated to 120° C. with stirring for 4 h. The solvent was removed in vacuo at 80° C., with subsequent additions and removals of toluene (2 mL) to eliminate DMF traces. The crude residue was purified by flash column chromatography to yield 37 (88 mg, 35%) as a cream solid.

¹H NMR (CDCl₃) δ 8.57 (s, 1H, 4-H), 6.33 (s, 1H, 5-H), 4.38 (t, 2H, J=6.7 Hz, 1′-CH₂), 2.73 (t, 2H, J=7.4 Hz, α-CH₂), 1.84 (qt, 2H, J=6.8 Hz, CH₂), 1.74 (m, 2H, CH₂), 1.50-1.26 (m, 18H, 9×CH₂), 0.94-0.85 (m, 6H, 2×CH₃); ¹³C NMR (CDCl₃) δ 168.9 (7a-C), 162.9 (2-C), 159.1 (6-C), 150.9 (4a-C), 99.5 (5-CH), 68.4 (1′-CH₂), 32.3 (CH₂), 30.0 (CH₂), 29.9 (CH₂), 29.7 (CH₂), 29.5 (CH₂), 28.9 (CH₂), 28.8 (CH₂), 28.7 (CH₂), 28.5 (CH₂), 27.6 (CH₂), 23.1 (CH₂), 22.9 (CH₂), 14.5 (CH₃), 14.4 (CH₃). Elemental analysis calcd for C₂₁H₃₄N₂O₂ (346.5): C 72.79, N 8.08, H 9.89; found C 73.68, N 10.03, H 8.06.

### 2-Cyclopentyloxy-6decyl-2,3-dihydrofuro[2,3-d]pyrimidine Cf2250

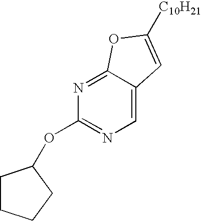

26 (1.00 g, 3.62 mmol), potassium carbonate (1.00 g, 7.24 mmol, 2 equiv.) and cyclopentyl bromide 39 (0.23 mL, 2.17 mmol, 2 equiv.) were suspended in dry DMF (15 mL) under N₂, and the mixture stirred at RT for 6 h. The grey/green suspension was then heated to 120° C. for 5 h, then allowed to cool with stirring overnight. The solvent was removed in vacuo at 80° C. The crude residue was purified by flash column chromatography in 0-1% MeOH/DCM eluent gradient to yield 40 as a white solid (0.87 g, 70% yield). ¹H NMR (CDCl₃) δ 8.48 (b, 1H, 4-H), 6.23 (s, 1H, 5-H), 5.36 (m, 1H, 1′-H), 2.65 (t, 2H, J=7.5 Hz, α-CH₂), 1.93-1.52 (m, 10H, 5×CH₂), 1.25-1.17 (m, 14H, 7×CH₂), 0.78 (t, 3H, J=6.5 Hz, CH₃); ¹³C NMR (CDCl₃) δ=168.8 (7a-C), 162.5 (2-C), 158.9 (6-C), 150.9 (4-CH), 113.9 (4a-C), 99.5 (5-CH), 80.3 (1′-CH), 33.1 (2×CH₂), 32.3 (CH₂), 30.0 (CH₂), 29.9 (CH₂), 29.7 (CH₂), 29.5 (CH₂), 28.7 (CH₂), 27.6 (2×CH₂), 24.2 (CH₂), 23.1 (CH₂), 14.5 (CH₃). Elemental analysis calculated for C₂₁H₃₂N₂O₂ (344.5): C 73.22, N 8.13, H 9.36; found C 73.85, N 8.61, H 9.84.

### 3-Cyclopentyl-6-decyl-2,3-dihydrofuro[2,3-d]pyrimidin-2-one Cf2251

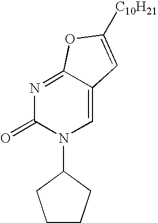

Also isolated from the above reaction was tde title compound 41 (0.18 g, 14%) as a yellow solid.

¹H NMR (CDCl₃) δ 7.79 (s, 1H, 4-H), 6.05 (s, 1H, 5-H), 5.16 (m, 1H, 1′-H), 2.56 (t, 2H, J=7.5 Hz, α-CH₂), 2.16 (m, 2H, CH₂), 1.82-1.55 (m, 8H, 4×CH₂), 1.25-1.19 (m, 14H, 7×CH₂), 0.80 (t, 3H, J=6.4 Hz, CH₃); ¹³C NMR (CDCl₃) δ 171.6 (7a-C), 160.3 (6-C), 156.2 (2-C), 135.8 (4-CH), 108.3 (4a-C), 99.0 (5-CH), 59.7 (1′-CH₂), 32.8 (2×CH₂), 32.3 (CH₂), 30.0 (CH₂), 29.7 (CH₂), 29.4 (CH₂), 28.7 (CH₂), 27.2 (2×CH₂), 24.5 (CH₂), 23.1 (CH₂), 14.5 (CH₃). Elemental analysis calculated for C₂₁H₃₂N₂O₂ (344.5): C 73.22, N 8.13, H 9.36; found C 72.83, N 8.18, H 9.84.

### 2-(1′-Ethyl-propyloxy)-6-decyl-2,3-dihydrofuro[2,3-d]pyrimidine Cf2252

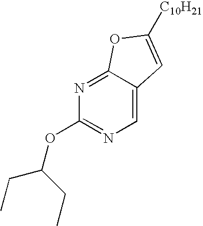

26 (0.50 g, 1.81 mmol), potassium carbonate (0.50 g, 3.62 mmol, 2 equiv) and 3-bromopentane 42 (0.45 mL, 3.62 mmol, 2 equiv.) were suspended in dry DMF (15 mL) under N₂, and the reaction mixture heated to 120° C. with stirring for 150 min. The dark suspension was allowed to cool to RT over 2 h, and then the solvent was removed under reduced pressure at 80° C. The residue was then subjected to flash column chromatography purification in a 0-5% MeOH/DCM eluent gradient to yield 43 as a yellow oil of weight 0.27 g (43% yield).

¹H NMR (CDCl₃) δ=8.44 (s, 1H, 4-H), 6.20 (s, 1H, 5-H), 4.96 (qt, 1H, J=6.0 Hz, 1′-H), 2.60 (t, 2H, J=7.5 Hz, α-CH₂), 1.68-1.55 (m, 6H, 3×CH₂), 1.24-1.13 (m, 12H, 6×CH₂), 0.84 (t, 6H, J=7.4 Hz, 2×CH₃), 0.74 (t, 3H, J=6.9 Hz, CH₃); ¹³C NMR (CDCl₃) δ=168.8 (7a-C), 162.9 (6-C), 158.7 (2-C), 150.9 (4-CH), 113.9 (4a-C), 99.5 (5-CH), 79.9 (1′-CH), 32.2 (CH₂), 29.9 (CH₂), 29.7 (CH₂), 29.6 (CH₂), 29.4 (CH₂), 28.7 (CH₂), 27.6 (CH₂), 26.7 (2×CH₂), 26.4 (CH₂), 23.0 (CH₂), 14.4 (CH₃), 9.9 (2×CH₃). Elemental analysis calcd for C₂₁H₃₄N₂O₂ (346.5): C 72.79, N 8.08, H 9.89; found C 73.12, N 8.56, H 9.93.

### 3-(1′-Ethyl-propyl)-6-decyl-2,3-dihydrofuro[2,3-d]pyrimidin-2-one Cf2253

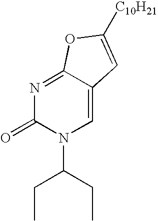

Also isolated from the above reaction was the title compound 44 as a white solid (0.168 g, 27%).

¹H NMR (CDCl₃) δ=7.72 (s, 1H, 4-H), 6.15 (s, 1H, 5-H), 4.94 (b, 1H, 1′-H), 2.67 (t, 2H, J=7.4 Hz, α-CH₂), 1.87 (m, 2H, CH₂), 1.71 (m, 4H, 2×CH₂), 1.36-1.23 (m, 14H, 7×CH₂), 0.91 (t, 9H, J=6.8 Hz, 3×CH₃); ¹³C NMR (CDCl₃) δ=171.2 (7a-C), 160.4 (6-C), 156.7 (2-C), 135.5 (4-CH), 108.3 (4a-C), 98.9 (5-CH), 32.3 (CH₂), 30.0 (CH₂), 29.9 (CH₂), 29.7 (2×CH₂), 29.5 (CH₂), 28.7 (CH₂), 28.0 (CH₂), 27.2 (CH₂), 23.1 (CH₂), 14.5 (CH₃), 10.8 (2×CH₃). Elemental analysis calculated for C₂₁H₃₄N₂O₂ (346.5): C 72.79, N 8.08, H 9.89; found C 72.65, N 8.16, H 10.08.

### 2-Cyclohexyloxy-6-decyl-2,3-dihydrofuro[2,3-d]pyrimidine Cf2294

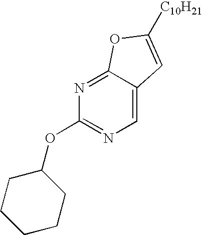

26 (300 mg, 1.086 mmol) and potassium carbonate (299 mg, 2.17 mmol, 2 equiv.) were suspended in dry DMF (10 mL) and cyclohexyl bromide 45 (0.54 mL, 2.17 mmol, 2 equiv.) added via syringe under N₂. The suspension was heated with stirring to 100 C overnight. The solvent was removed in vacuo at 80° C. The residue was suspended in DCM and washed with water. The organic layer was dried over MgSO₄, the solvent distilled in vacuo and the resultant residue purified by flash column chromatography in a 0-2% MeOH/DCM eluent gradient to yield 46 as a clear colourless waxy solid (78 mg, 20% yield).

¹H NMR (CDCl₃) δ=8.68 (s, 1H, 4-H), 6.43 (s, 1H, 5-H), 5.16 (m, 1H, 1′-H), 2.85 (t, 2H, J=7.4 Hz, α-CH₂), 2.19 (m, 2H, CH₂), 1.94 (m, 2H, CH₂), 1.84 (m, 2H, CH₂), 1.72 (m, 2H, CH₂), 1.58-1.32 (m, 18H, 9×CH₂), 0.99 (t, 3H, J=6.4 Hz, CH₃); ¹³C NMR (CDCl₃) δ 168.9 (7a-C), 162.3 (2-C), 158.9 (6-C), 151.0 (4-CH), 114.0 (4a-C), 99.6 (5-CH), 75.8 (1′-CH), 32.3 (CH₂), 32.0 (CH₂), 30.0 (CH₂), 29.9 (CH₂), 29.7 (2×CH₂), 29.5 (CH₂), 28.8 (CH₂), 27.6 (CH₂), 26.0 (CH₂), 24.3 (2×CH₂), 23.1 (CH₂), 14.6 (CH₃).

### 3-Cyclohexyl-6-decyl-2,3-dihydrofuro[2,3-d]pyrimidin-2-one Cf2295

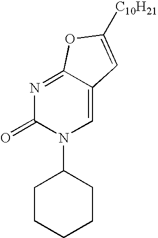

Also isolated from the above reaction was the title compound 47 (23 mg, 6%) as a white solid. ¹H NMR (CDCl₃) 7.86 (s, 1H, 4-H), 6.13 (s, 1H, 5-H), 4.90 (m, 1H, 1′-H), 2.68 (t, 2H, J=7.4 Hz, α-CH₂), 2.09-1.30 (m, 26H, 13×CH₂), 0.93 (t, 3H, J=6.2 Hz, CH₃); ¹³C NMR (CDCl₃) δ 171.5 (7a-C), 160.3 (2-C), 155.8 (6-H), 135.6 (4-CH), 108.1 (4a-C), 98.9 (5-CH), 57.1 (1′-CH), 33.3 (CH₂), 32.3 (CH₂), 32.0 (2×CH₂), 29.7 (2×CH₂), 26.2 (CH₂), 25.8 (CH₂), 24.3 (CH₂), 23.1 (CH₂), 14.6 (CH₃).

### 6-Decyl-3-(tetrahydro-furan-2-ylmethyl)-3H-furo[2,3-d]pyrimidin-2-one 72 Cf2309

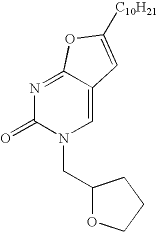

The title compound 72 (157 mg, 42%) was also isolated from the reaction mixture as a white solid.

¹H NMR (CDCl₃) δ 7.95 (s, 1H, 4-H), 6.13 (s, 1H, 5-H), 4.55 (dd, J=2.3, 13.6 Hz, 1H, N—CH₂-THF), 4.29 (m, 1H, N—CH₂-THF), 3.93-3.72 (m, 3H, THF-CH), 2.68 (t, J=7.4 Hz, 2H, 1′-CH₂), 2.26-2.15 (m, 1H, THF-CH), 2.00-1.90 (m, 2H, CH₂), 1.71-1.63 (m, 3H, THF-CH), 1.37-1.31 (m, 14H, CH₂), 0.93 (t, J=6.4 Hz, 3H, CH₃); ¹³C NMR (CDCl₃) δ 172.4 (7a-C), 160.2 (2-C), 156.1 (6-C), 140.5 (4-CH), 107.9 (4a-C), 98.9 (5-CH), 77.3 (THF-C), 68.6 (THF-C), 54.9 (N-1′-CH₂-THF), 32.3 (CH₂), 30.0 (CH₂), 29.9 (CH₂), 29.8 (CH₂), 29.7 (CH₂), 29.5 (CH₂), 29.2 (CH₂), 28.7 (CH₂), 27.2 (CH₂), 26.2 (CH₂), 23.1 (CH₂), 14.6 (—CH₂CH₃).

### 2-Cyclohexylmethoxy-6-decyl-furo[2,3-d]pyrimidine Cf2274

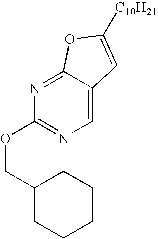

26 (0.30 g, 1.086 mmol) and potassium carbonate (0.30 g, 2.17 mmol, 2 equiv) were suspended in dry DMF (10 mL) under N₂, and (bromomethyl)cyclohexane 48 (0.30 mL, 2.17 mmol, 2 equiv.) added via syringe to the resultant stirred suspension. The suspension was then heated to 120° C. with stirring for 3 h, then allowed to cool with stirring overnight. The solvent was then removed in vacuo at 80° C., and the crude mixture purified by flash chromatography in a 0-2% methanol/DCM eluent gradient to yield 49 (189 mg, 47%) as white solid.

¹H NMR (CDCl₃) δ 8.63 (s, 1H, 4-H), 6.67 (s, 1H, 5-H), 4.35 (d, J=6.2 Hz, 2H, O—CH₂-CyHx), 2.79 (t, J=7.4 Hz, 2H, 1′-CH₂), 1.97-1.90 (m, 3H, CyHx-CH), 1.78 (m, 6H, CyHx-CH), 1.38-1.31 (m, 16H, CH₂), 1.19-1.08 (m, 2H, CyHx-CH), 0.91 (t, J=6.4 Hz, 3H, —CH₂CH₃); ¹³C NMR (CDCl₃) δ 168.9 (7a-C), 163.0 (2-C), 159.1 (6-C), 150.9 (4-CH), 114.2 (4a-C), 99.5 (5-CH), 73.6 (O—CH₂-CyHx), 37.7 (CyHx-C), 32.3 (1′-CH₂), 30.2 (CyHx-C), 30.0 (2×CH₂), 29.8 (CH₂), 29.7 (CH₂), 29.5 (CH₂), 28.8 (CH₂), 27.6 (CH₂), 26.9 (CH₂), 26.2 (2×CH₂), 23.1 (CH₂), 14.6 (—CH₂CH₃).

### 3-Cyclohexylmethyl-6-decyl-3H-furo[2,3-d]pyrimidin-2-one Cf2275

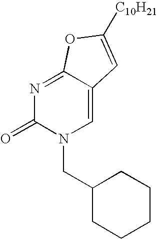

Also isolated from the mix as a white solid in a yield of 33% (129 mg) was the title compound 50.

¹H NMR (CDCl₃) δ 7.72 (s, 1H, 4-H), 6.12 (s, 1H, 5-H), 3.64 (d, J=7.3 Hz, 2H, N—CH₂-CyHx), 2.66 (t, J=7.5 Hz, 2H, 1′-CH₂), 2.04-1.95 (m, 1H, CyHx-CH), 1.94-1.68 (m, 6H, CyHx-CH), 1.35-1.29 (m, 16H, CH₂), 1.23 (m, 2H, CyHx-CH), 1.02 (m, 2H, CyHx-CH), 0.90 (t, J=6.4 Hz, 3H, —CH₂CH₃); ¹³C NMR (CDCl₃) δ 172.3 (7a-C), 160.3 (2-C), 156.0 (6-C), 139.7 (4-CH), 107.7 (4a-C), 98.7 (5-CH), 58.8 (N—CH₂-CyHx), 36.9 (CyHx-C), 32.3 (1′-CH₂), 30.9 (CyHx-C), 30.0 (CH₂), 29.9 (CH₂), 29.8 (CH₂), 29.6 (CH₂), 29.5 (CH₂), 28.7 (CH₂), 27.2 (CH₂), 26.6 (CH₂), 26.0 (2×CH₂), 23.1 (CH₂), 14.6 (—CH₂CH₃).

### 2-Benzyloxy-6-decyl-furo[2,3-d]pyrimidine Cf2307

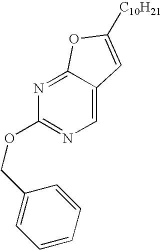

26 (0.30 g, 1.086 mmol), potassium carbonate (0.30 g, 2.17 mmol, 2 equiv) and benzyl chloride (51, 0.25 mL, 2.17 mmol, 2 equiv.) were suspended in dry DMF (5 mL) under N₂, and the reaction mixture heated to 100 C with stirring overnight. The solvent was then removed in vacuo at 80° C., and the crude mixture purified by flash chromatography in a 0-5% methanol/DCM eluent gradient to yield 52 (54 mg, 14%) as white solid.

¹H NMR (CDCl₃) δ 8.66 (s, 1H, 4-H), 7.57 (d, J=6.7 Hz, 2H, Ar—CL), 7.36 (m, 3H, Ar—CH), 6.40 (s, 1H, 5-H), 5.54 (s, 2H, O—CH₂-Ph), 2.78 (t, J=7.2 Hz, 2H, 1′-CH₂), 1.79 (m, 2H, CH₂), 1.32 (m, 14H, CH₂), 0.92 (m, 3H, —CH₂CH₃); ¹³C NMR (CDCl₃) δ 168.8 (7a-C), 162.5 (2-C), 159.4 (6-C), 150.9 (4-CH), 137.0 (Ar—C), 128.8 (Ar—C), 128.4 (Ar—C), 128.3 (Ar—C), 113.9 (4a-C), 99.6 (5-CH), 69.7 (O—CH₂-Ph), 32.3 (1′-CH₂), 30.0 (CH₂), 29.9 (CH₂), 29.7 (2×CH₂), 29.5 (CH₂), 28.8 (CH₂), 27.6 (CH₂), 23.1 (CH₂), 14.6 (—CH₂CH₃).

### 3-Benzyl-6-decyl-3H-furo[2,3-d]pyrimidin-2-one Cf2308

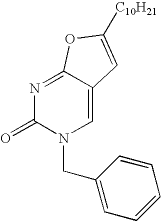

Also isolated from the crude residue was the title compound 53 (258 mg, 65%) as white solid.

¹H NMR (CDCl₃) δ 7.74 (s, 1H, 4-H), 7.42 (m, 5H, Ar—CH), 6.07 (s, 1H, 5-H), 5.26 (s, 2H, N—CH₂-Ph), 2.67 (t, J=7.3 Hz, 2H, 1′-CH₂), 1.83 (m, 2H, CH₂), 1.66 (m, 14H, CH₂), 0.93 (t, J=6.9 Hz, 3H, —CH₂CH₃); ¹³C NMR (CDCl₃) δ 172.3 (7a-C), 160.8 (2-C), 156.1 (6-C), 138.3 (4-CH), 135.9 (Ar—C), 129.6 (Ar—C), 129.1 (Ar—C), 129.0 (Ar—C), 108.6 (4a-C), 98.8 (5-CH), 54.4 (N—CH₂-Ph), 32.3 (1′-CH₂), 30.0 (CH₂), 29.9 (CH₂), 29.7 (CH₂), 29.6 (CH₂), 29.4 (CH₂), 28.7 (CH₂), 27.2 (CH₂), 23.1 (CH₂), 14.5 (—CH₂CH₃).

### 6-Decyl-3-(tetrahydro-furan-2′-yl)-2,3-dihydrofuro[2,3-d]pyrimidin-2-one Cf2249

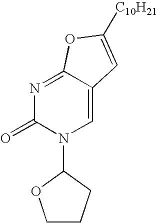

26 (0.30 g, 0.19 mmol) and a catalytic amount of DMAP were suspended in dry DMF (8 mL) under an atmosphere of N₂, and 2-tert-butoxytetrahydrofuran 54 (0.34 mL, 0.31 g, 2.17 mmol, 2 equiv.) added via syringe with stirring. The resultant green suspension was heated to 150° C. for 5 h with stirring, then the solvent was removed under reduced pressure at 80° C. The residue was purified via flash column chromatography in DCM to yield 90 mg (24%) of the title compound 55 as a pale yellow compound.

¹H NMR (CDCl₃) δ 7.95 (s, 1H, 4-H), 6.10 (m, 2H, 5-H and 2′-H), 4.29 (m, 1H, 5′-H), 4.06 (m, 1H, 5′-H), 2.63 (t, 2H, J=7.5 Hz, α-CH₂), 2.56 (m, 1H, THF-CH), 2.17 (m, 1H, THF-CH), 2.01 (m, 1H, THF-CH), 1.83 (m, 1H, THF-CH), 1.66 (m, 2H, CH₂), 1.30-1.1.9 (m, 14H, 7×CH₂), 0.86 (t, 3H, J=6.3 Hz, CH₃); ¹³C NMR (CDCl₃) δ 171.9 (7a-C), 160.0 (6-C), 155.2 (2-C), 134.2 (4-CH), 107.6 (4a-C), 99.1 (5-CH), 90.2 (2′-CH), 71.1 (5′-CH₂), 33.8 (CH₂), 32.3 (CH₂), 30.0 (CH₂), 29.9 (CH₂), 29.7 (2×CH₂), 29.5 (CH₂), 28.7 (CH₂), 27.2 (CH₂), 23.7 (CH₂), 23.1 (CH₂), 14.6 (CH₃).

### Methanesulfonic acid tetrahydro-furan-3-yl ester 64

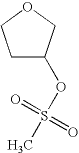

3-Hydroxytetrahydrofuran 57 (0.50 g, 0.46 mL, 5.5 mmol) and triethylamine (1 mL, 7 mmol, 1.3 equiv.) were dissolved in dry DCM (5 mL) and the solution cooled to 0° C. with stirring. Methanesulfonyl chloride 63 (0.55 mL, 7 mmol, 1.3 equiv.) was added slowly via syringe to the chilled solution. The solution was allowed to warm to RT, and the resultant suspension stirred at RT for 24 h. Dry DCM (20 mL) was then added to the suspension to re-form a solution. The solution was allowed to stir at RT for a further 36 h. The solvent was removed in vacuo and the residue dissolved in water. The aqueous solution was extracted with DCM. The DCM extracts were then washed with brine, and the brine washings extracted with fresh DCM. The combined organic layers were then dried over MgSO₄. The solvent was removed under reduced pressure to yield 64 as a yellow viscous liquid (0.80 g, 96%), which was used without further purification.

¹H NMR (CDCl₃) δ 5.20 (m, 1H, 1′-CH), 3.94-3.74 (m, 4H, THF-CH), 2.96 (s, 3H, CH₃), 2.18-2.11 (m, 2H, THF-CH); ¹³C NMR (CDCl₃): δ1.38 (1′-CH), 73.4 (2′-CH₂), 67.1 (4′-CH₂), 38.8 (CH₃), 33.7 (3′-CH₂).

### 6-Decyl-2-(tetrahydro-furan-3-yloxy)-furo[2,3-d]pyrimidine 58

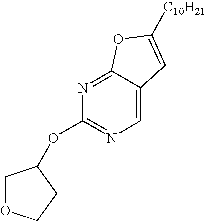

26 (0.182 g, 0.66 mmol), potassium carbonate (0.182 g, 1.33 mmol, 2 equiv) and methanesulfonic acid tetrahydro-furan-3-yl ester 64 (0.105 g, 0.63 mmol, 0.95 equiv) were suspended in dry DMF (5 mL) under N₂, and the reaction mixture heated to 80° C. with stirring for 8 h. The solvent was then removed in vacuo at 80° C., and the resultant residue purified by flash chromatography in a 0-5% methanol/DCM eluent gradient to yield 58 (140 mg, 62%) as white solid.

¹H NMR (CDCl₃) δ 8.64 (s, 1H, 4-H), 6.40 (s, 1H, 5-H), 5.64-5.59 (m, 1H, O-1′-THF), 4.07-3.96 (m, 4H, THF-CH), 2.80 (t, J=7.5 Hz, 2H, 1′-CH₂), 1.79 (quin, J=7.6 Hz, 2H, CH₂), 1.39-1.31 (m, 14H, CH₂), 0.93 (t, J=6.5 Hz, 3H, —CH₂CH₃); ¹³C NMR (CDCl₃) δ 168.6 (7a-C), 163.0 (2-C), 159.5 (6-C), 151.0 (4-CH), 114.6 (4a-C), 99.6 (5-CH), 78.2 (1′-THF-C), 78.2 (THF-C), 73.8 (THF-C), 67.7 (1′-THF-C), 33.5 (1′-CH₂), 32.3 (CH₂), 30.0 (CH₂), 29.9 (CH₂), 29.8 (CH₂), 29.7 (CH₂), 29.5 (CH₂), 28.8 (CH₂), 27.6 (CH₂), 23.1 (CH₂), 14.6 (—CH₂CH₃).

### 6-Decyl-3-(tetrahydro-furan-3-yl)-3H-furo[2,3-d]pyrimidin-None Cf2276

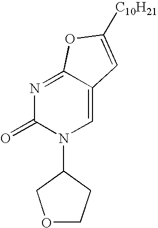

Also isolated from the residue was the title compound 59 as a white solid (22 mg, 10%).

¹H NMR (CDCl₃) δ 8.00 (s, 1H, 4-H), 6.12 (s, 1H, 5-H), 5.68 (m, 1H, N-1′-THF), 4.23-4.09 (m, 2H, THF-CH), 3.97-3.86 (m, 2H, THF-CH), 2.68 (m, 2H, 1′-CH₂), 1.72 (m, 2H, CH₂), 1.36-1.30 (m, 16H, CH₂), 0.91 (t, J=6.3 Hz, 3H, —CH₂CH₃); ¹³C NMR (CDCl₃) δ 171.8 (7a-C), 160.7 (2-C), 156.0 (6-C), 136.0 (4-CH), 109.1 (4a-C), 99.1 (5-CH), 73.4 (1′-THF-C), 78.2 (THF-C), 67.6 (THF-C), 58.1 (1′-THF-C), 34.2 (1′-CH₂), 32.3 (CH₂), 30.0 (CH₂), 29.9 (CH₂), 29.7 (CH₂), 29.6 (CH₂), 29.4 (CH₂), 28.7 (CH₂), 27.2 (CH₂), 23.1 (CH₂), 14.5 (—CH₂CH₃).

### Methanesulfonic acid tetrahydrofuran-3-yl methyl ester 66

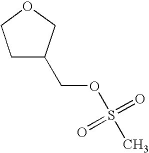

Tetrahydro-3-furan methanol 65 (0.50 g, 4.9 mmol) was dissolved in dry DCM (30 mL) and triethylamine (1.06 mL, 8.8 mmol, 1.8 equiv) was added to the solution via syringe under N₂ with stirring. The solution was cooled to 0° C. and methanesulfonyl chloride 63 (0.68 mL, 8.8 mmol, 1.8 equiv) added dropwise via syringe. The resultant solution was allowed to warm to RT and stirred at RT for 36 h. The solvent was then removed in vacuo. The residue was dissolved in fresh DCM and water (25 mL) added to the solution. The solution was then extracted with DCM. The DCM extracts were washed with brine, and the brine back-extracted with DCM. The combined DCM extracts were then reduced in vacuo to yield a yellow oil (66, 0.88 g, quantitative).

### Methanesulfonic acid tetrahydro-furan-2-yl methyl ester 70

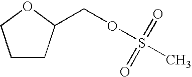

Tetrahydrofurfuryl alcohol 69 (0.50 g, 4.9 mmol) was dissolved in dry DCM (30 mL) and triethylamine (1.06 mL, 8.8 mmol, 1.8 equiv) was added to the solution via syringe under N₂ with stirring. The solution was cooled to 0° C. and methanesulfonyl chloride 63 (0.68 mL, 8.8 mmol, 1.8 equiv) added dropwise via syringe to the cooled solution. The resultant solution was allowed to warm to RT and stirred at RT for 36 h. The solvent was then removed in vacuo. The residue was dissolved in fresh DCM and water (25 mL) added to the solution. The solution was then extracted with DCM. The DCM extracts were washed with brine, and the brine back-extracted with DCM. The combined DCM extracts were dried (MgSO₄), then reduced in vacuo to yield a yellow oil (70, 0.86 g, 98%).

### 6-Decyl-2-(tetrahydro-furan-2-ylmethoxy)-furo[2,3-d]pyrimidine 71

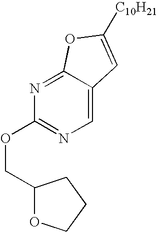

26 (0.182 g, 1.086 mmol), potassium carbonate (0.182 g, 2.17 mmol, 2 equiv) and methanesulfonic acid tetrahydro-furan-2-ylmethyl ester 70 (0.186 g, 1.086 mmol) were suspended in dry DMF (5 mL) under N₂, and the reaction mixture heated to 100° C. with stirring under N₂ for 8 h. The solvent was removed in vacuo. The resultant residue was suspended in water (100 mL) and extracted with DCM (5×50 mL), then washed with brine. The combined DCM extracts were dried over MgSO₄, filtered, reduced in vacuo and purified by flash column chromatography in a carefully altered 0-5% methanol/DCM solvent eluent gradient to yield 120 mg (32%) of the title compound 71 as a white solid.

¹H NMR (CDCl₃) δ 8.63 (s, 1H, 4-H), 6.39 (s, 1H, 5-H), 4.49-4.36 (m, 3H, THF-CH), 4.03-3.94 (m, 1H, O—CH₂-THF), 3.91-3.84 (m, 1H, O—CH₂-THF), 2.79 (t, J=7.4 Hz, 2H, 1′-CH₂), 2.19-1.84 (m, 4H, THF-CH), 1.80-1.73 (m, 2H, CH₂), 1.38-1.31 (m, 14H, CH₂), 0.93 (t, J=6.4 Hz, 3H, CH₃); ¹³C NMR (CDCl₃) δ 168.8 (7a-C), 162.6 (2-C), 159.3 (6-C), 150.9 (4-CH), 114.5 (4a-C), 99.5 (5-CH), 77.6 (THF-C), 70.1 (THF-C), 68.9 (O-1′-CH₂-THF), 32.3 (CH₂), 30.0 (CH₂), 29.9 (CH₂), 29.7 (2×CH₂), 29.5 (CH₂), 28.8 (CH₂), 28.7 (CH₂), 27.6 (CH₂), 26.1 (CH₂), 23.1 (CH₂), 14.6 (—CH₂CH₃).

### 6-Decyl-3-(tetrahydro-furan-2-ylmethyl)-3H-furo[2,3-d]pyrimidin-2-one 72

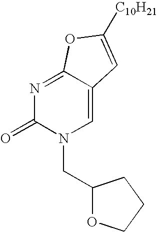

The title compound 72 (157 mg, 42%) was also isolated from the reaction mixture as a white solid. ¹H NMR (CDCl₃) δ 7.95 (s, 1H, 4-H), 6.13 (s, 1H, 5-H), 4.55 (dd, J=2.3, 13.6 Hz, 1H, N—CH₂-THF), 4.29 (m, 1H, N—CH₂-THF), 3.93-3.72 (m, 3H, THF-CH), 2.68 (t, J=7.4 Hz, 2H, 1′-CH₂), 2.26-2.15 (m, 11H, THF-CR), 2.00-1.90 (m, 2H, CH₂), 1.71-1.63 (m, 3H, THF-CR), 1.37-1.31 (m, 14H, CH₂), 0.93 (t, J=6.4 Hz, 3H, CH₃); ¹³C NMR (CDCl₃) δ 172.4 (7a-C), 160.2 (2-C), 156.1 (6-C), 140.5 (4-CU, 107.9 (4a-C), 98.9 (5-CU), 77.3 (THF-C), 68.6 (THF-C), 54.9 (N-1′-CH₂-THF), 32.3 (CH₂), 30.0 (CH₂), 29.9 (CH₂), 29.8 (CH₂), 29.7 (CH₂), 29.5 (CH₂), 29.2 (CH₂), 28.7 (CH₂), 27.2 (CH₂), 26.2 (CH₂), 23.1 (CH₂), 14.6 (—CH₂CH₃).

### 6-Decyl-2-(tetrahydro-pyran-2-ylmethoxy)-furo[2,3-d]pyrimidine 61

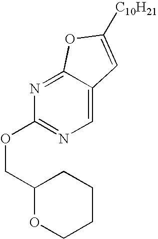

26 (0.30 g, 1.086 mmol), potassium carbonate (0.30 g, 2.17 mmol, 2 equiv) were suspended in dry DMF (5 mL) under N₂, and 2-(bromomethyl)tetrahydro-2H-pyran 74 (0.28 mL, 2.17 mmol, 2 equiv) added via syringe with stirring under N₂. The resultant mixture was heated to 110° C. with stirring overnight. The solvent was then removed in vacuo at 80° C., and the residue suspended in water (100 mL) and extracted with DCM (5×50 mL). The combined DCM extracts were washed with brine, dried over MgSO₄, filtered, reduced in vacuo and purified slowly by flash chromatography in a DCM, then carefully altered 0-5% methanol/DCM eluent gradient to yield the title compound 61 as a white solid (120 mg, 30%) as a white solid. ¹H NMR (CDCl₃) δ 8.63 (s, 1H, 4-H), 6.38 (s, 1H, 5-H), 4.50-4.34 (m, 2H, O—CH₂-THP), 4.08 (m, 1H, THP-CH), 3.83 (m, 1H, THP-CL), 3.54 (t, J=11.3 Hz, 1H, THP-CL), 2.79 (t, J=7.4 Hz, 2H, 1′-CH₂), 1.97-1.94 (m, 1H, THP-CH), 1.76 (app d, J=7.6 Hz, 2H, CH₂), 1.39-1.31 (m, 16H, CH₂), 0.93 (t, J=6.5 Hz, 3H, —CH₂CH₃); ¹³C NMR (CDCl₃) δ 168.8 (7a-C), 162.6 (2-C), 159.3 (6-C), 150.9 (4-CH), 114.5 (4a-C), 99.5 (5-CH), 76.0 (THP-C), 71.3 (THP-C), 67.7 (1′-CH₂-THP), 32.3 (CH₂), 30.0 (CH₂), 29.9 (CH₂), 29.8 (CH₂), 29.7 (CH₂), 29.5 (CH₂), 28.8 (CH₂), 28.5 (CH₂), 27.6 (CH₂), 26.3 (CH₂), 23.5 (CH₂), 23.1 (CH₂), 14.6 (—CH₂CH₃).

### 6-Decyl-3-(tetrahydro-pyran-2-ylmethyl)-3H-furo[2,3-d]pyrimidin-2-one 62

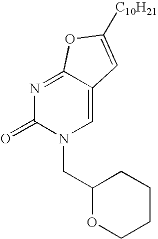

Also isolated from the mixture was 62, the title compound in 26% yield (105 mg) as a white compound.

¹H NMR (CDCl₃) δ 7.84 (s, 1H, 4-H), 6.11 (s, 1H, 5-H), 4.48 (dd, J=1.9, 6.7 Hz, 1H, N—CH₂-THP), 3.92 (app d, J=10.7 Hz, 1H, THP-CH), 3.71 (m, 1H, THP-CH), 3.52 (app q, J=4.5, 6.7 Hz, 1H, THP-CH), 3.38-3.30 (m, 1H, THP-CH), 2.66 (t, J=7.4 Hz, 2H, 1′-CH₂), 1.97-1.94 (m, 1H, THP-CH), 1.88-1.47 (m, 4H, CH₂), 1.35-1.29 (m, 18H, CH₂), 0.91 (t, J=6.5 Hz, 3H, —CH₂CH₃); ¹³C NMR (CDCl₃) δ 172.5 (7a-C), 160.0 (2-C), 156.1 (6-C), 141.1 (4-CH), 107.5 (4a-C), 98.9 (5-CH), 75.3 (THP-C), 68.7 (THP-C), 56.6 (1′-CH₂-THP), 32.3 (CH₂), 30.0 (CH₂), 29.9 (CH₂), 29.8 (CH₂), 29.7 (CH₂), 29.5 (CH₂), 29.4 (CH₂), 28.7 (CH₂), 27.2 (CH₂), 26.3 (CH₂), 23.3 (CH₂), 23.1 (CH₂), 14.6 (—CH₂CH₃).

### Methanesulfonic acid 3-methyl-cyclopentyl ester 76

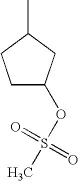

3-Methylcyclopentanol 75 (0.5 g, 4.99 mmol) was dissolved in dry DCM (25 mL), and triethylamine (0.8 mL, 6.5 mmol, 1.3 equiv) added to the stirred solution under N₂, which was then cooled to 0° C. Methanesulfonyl chloride (0.5 mL, 6.5 mmol, 1.3 equiv) was added dropwise via syringe to the chilled solution, the resultant solution warmed to RT and allowed to react at RT with stirring for 36 h. The solvent was removed in vacuo, and the residue dissolved in water (50 mL), which was extracted with DCM (5×50 mL). The combined DCM extracts were washed with brine (which was back extracted with fresh DCM (25 mL)), dried (MgSO₄), filtered and reduced under vacuum to yield a clear yellow oil (789 mg, 88%).

### 6-Decyl-2-(4-methoxybenzyloxy)-3H-furo[2,3-a]pyrimidine Cf2315

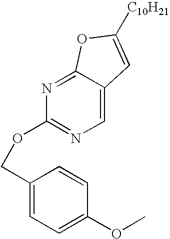

6-Decyl-2,3-dihydrofuro[2,3-d]pyrimidin-2-one 26 (0.50 g, 1.81 mmol) and potassium carbonate (0.50 g, 3.62 mmol, 2 equiv.) were suspended in dry DMF (6 mL), and 4-methoxybenzyl chloride (0.5 mL, 3.62 mmol, 2 equiv) added to the stirred solution via syringe under N₂. The resultant mixture was heated with stirring to 120° C. overnight. The solvent were removed in vacuo at 80° C., then the residue purified by flash column chromatography in a 0-5% MeOH/DCM eluent gradient to yield the title compound X (63 mg, 9%) as a white solid.

¹H NMR (CDCl₃) δ 8.61 (s, 1H, H-4), 7.48 (d, J=8.4 Hz, 2H, Ar—CH), 6.93 (d, J=8.7 Hz, 2H, Ar—CH), 6.35 (s, 1H, H-5), 5.44 (s, 2H, Ph-CH₂), 3.82 (s, 3H, O—CH₃), 2.77 (t, J=7.3 Hz, 2H, α-CH₂), 1.75 (qt, J=7.3 Hz, 2H, CH₂), 1.40-1.29 (m, 14H, CH₂), 0.91 (t, J=7.0 Hz, 3H, CH₃); ¹³C NMR (CDCl₃) δ 168.2 (7a-C), 159.6 (C-2), 159.3 (C-6), 149.9 (4-CH), 130.8 (Ar—CH), 129.7 (Ar—CH), 116.2 (Ar—CH, 114.3 (Ar—CH), 99.7 (5-CH), 69.7 (Ph-CH₂), 32.3 (α-CH₂), 30.0 (CH₂), 29.9 (CH₂), 29.7 (CH₂), 29.6 (CH₂), 28.8 (CH₂), 27.6 (CH₂), 23.5 (CH₂), 21.1 (CH₂), 14.6 (CH₃).

### 6-Decyl-3-(4-methoxybenzyl)-3H-furo[2,3-d]pyrimidin-2-one Cf2316

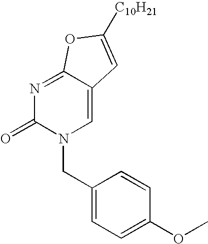

Also obtained from the mixture was the title compound as a white solid 34 (312 mg, 44%).

¹H NMR (CDCl₃) δ7.70 (s, 1H, H-4), 7.35 (d, J=8.0 Hz, 2H, Ar—CR), 6.95 (d, J=7.8 Hz, 2H, Ar—CR), 6.06 (s, 1H, H—S), 5.18 (s, 2H, Ph-CH₂), 3.86 (s, 3H, O—CH₃), 2.66 (t, J=7.5 Hz, 2H, α-CH₂), 1.69 (m, 2H, CH₂), 1.40-1.31 (m, 14H, CH₂), 0.93 (t, J=7.2 Hz, 3H, CH₃); ¹³C NMR (CDCl₃) δ 172.4 (7a-C), 160.6 (C-2), 155.8 (C-6), 138.1 (4-CH), 130.7 (Ar—CH), 128.5 (Ar—CH), 114.9 (Ar—CH), 108.2 (4a-C), 98.9 (5-CH), 55.7 (O—CH₃), 54.0 (Ph-CH₂), 32.3 (α-CH₂), 30.0 (CH₂), 29.9 (CH₂), 29.7 (CH₂), 29.6 (CH₂), 29.4 (CH₂), 28.7 (CH₂), 27.2 (CH₂), 23.1 (CH₂), 14.6 (CH₃).

### 6-Decyl-2-(4-methylbenzyloxy)-3H-furo[2,3-d]pyrimidine Cf2313

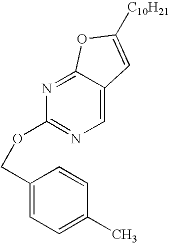

6-Decyl-2,3-dihydrofuro[2,3-d]pyrimidin-2-one 26 (0.50 g, 1.81 mmol), potassium carbonate (0.50 g, 3.62 mmol, 2 equiv) were suspended in dry DMF (5 ml) and 4-methylbenzyl chloride (0.5 mL, 3.62 mmol, 2 equiv) added to the stirred suspension under N₂ via syringe. The resultant mixture was then heated at 100° C. overnight. The solvents were removed in vacuo at 80° C. and the resultant residue purified by flash column chromatography in a 0-5% methanol/DCM eluent gradient to yield 30, the title product (105 mg, 15%), as a white solid.

¹H NMR (CDCl₃) δ 8.64 (s, 11H, H-4), 7.45 (d, J=7.9 Hz, 2H, Ar—CO), 7.21 (d, J=8.0 Hz, 2H, Ar—CH), 6.40 (s, 11H, H-5), 5.49 (s, 2H, Ph-CH₂), 2.80 (t, J=7.4 Hz, 2H, α-CH₂), 2.43 (s, 3H, Ar—CH₃), 1.79 (qt, J=6.8 Hz, 2H, CH₂), 1.47-1.32 (m, 14H, CH₂), 0.94 (t, J=7.2 Hz, 3H, CH₃); ¹³C NMR (CDCl₃) δ 168.8 (7a-C), 162.2 (C-2), 159.3 (C-6), 149.9 (4-CH), 138.5 (Ar—CH), 129.5 (Ar—CH), 128.5 (Ar—CH), 114.3 (Ar—CH), 99.6 (5-CH), 69.6 (Ph-CH₂), 32.3 (α-CH₂), 30.0 (CH₂), 29.9 (CH₂), 29.7 (CH₂), 29.5 (CH₂), 28.8 (CH₂), 23.1 (CH₂), 21.7 (CH₂), 14.6 (CH₃).

### 6-Decyl-3-(4-methylbenzyl)-3H-furo[2,3-d]pyrimidin-2-one Cf2314

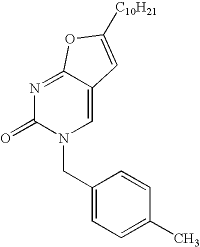

Also obtained from the mixture was the title compound 31 (440 mg, 65%) as a white solid.

¹H NMR (CDCl₃) δ 7.71 (s, 1H, H-4), 7.30 (d, J=8.2 Hz, 2H, Ar—CH), 7.23 (d, J=8.0 Hz, 2H, Ar—CH), 6.05 (s, 1H, H-5), 5.20 (s, 2H, Ph-CH₂), 2.66 (t, J=7.4 Hz, 2H, α-CH₂), 2.63 (s, 3H, Ar—CH₃), 1.73 (qt. J=7.6 Hz, 2H, CH₂), 1.43-1.32 (m, 14H, CH₂), 0.92 (t, J=7.0 Hz, 3H, CH₃); ¹³C NMR (CDCl₃) δ 172.3 (7a-C), 160.6 (C-2), 156.2 (C-6), 138.9 (4-CH), 132.8 (Ar—CH), 129.2 (Ar—CH), 128.5 (Ar—CH), 114.3 (Ar—CH), 98.8 (5-CH), 54.2 (Ph-CH₂), 32.3 (α-CH₂), 30.0 (CH₂), 29.9 (CH₂), 29.7 (CH₂), 29.6 (CH₂), 29.4 (CH₂), 28.7 (CH₂), 27.1 (CH₂), 23.1 (CH₂), 21.6 (CH₃), 14.6 (CH₃).

### 6-Hexyl-2,3-dihydrofuro[2,3-d]pyrimidin-2-one

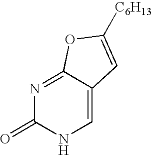

5-Iodouracil 23 (5.00 g, 21 mmol), tetrakis(triphenylphosphine)palladium(0) (1.0 g, 0.87 mmol, 0.04 equiv), and copper iodide (0.80 g, 4.2 mmol, 0.2 equiv) were dissolved in dry DMF (50 mL) with stirring under N₂. DIPEA (7.3 mL, 5.42 g, 42 mmol, 2 equiv), then 1-octyne (9.3 mL, 6.93 g, 63 mmol, 3 equiv) were added sequentially to the solution via syringe and the resultant solution, which darkened from golden to dark green over 20 min, left to stir at RT for 18 h. A further addition of copper iodide (0.80 g) was then made, followed by triethylamine (25 mL) and the resultant suspension heated at 120° C. for 6 h. The suspension was allowed to cool, the volume of solvent reduced to ca. 20 mL, and the solid collected by filtration, washed with DCM and methanol to yield a grey powder of weight 3.13 g (38, 68%). ¹H NMR (CDCl₃) δ 12.23 (br, 1H, NH), 8.16 (br, 1H, H-4), 6.38 (br, 1H, H-5), 2.65 (t, J=7.1 Hz, 2H, x-CH₂), 1.63 (qt, J=7.4 Hz, 2H, CH₂), 1.31 (m, 6H, CH₂), 0.88 (t, J=6.4 Hz, 3H, CH₃).

### 6-Hexyl-3-methyl-3H-furo[2,3-d]pyrimidin-2-one Cf2344

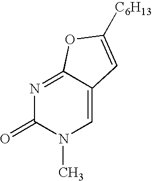

6-Hexyl-2,3-dihydrofuro[2,3-d)pyrimidin-2-one 38 (0.40 g, 1.82 mmol) and potassium carbonate (0.50 g, 3.64 mmol, 2 equiv) were suspended in dry DMF (5 mL) under N₂ and methyl iodide (0.23 mL, 3.64 mmol, 2 equiv) added via syringe to the stirred suspension, which was then heated to 80° C. overnight. The solvents were removed in vacuo and the crude purified by flash column chromatography in a 0-5% MeOH/DCM solvent gradient to yield the title product 40 as a white solid in very low yield (25 mg, 6%).

¹H NMR (CDCl₃) δ 7.76 (s, 1H, H-4), 6.04 (s, 1H, H-5), 3.59 (s, 3H, N—CH₃), 2.59 (t, J=7.5 Hz, 2H, α-CH₂), 1.63 (qt, J=7.4 Hz, 2H, CH₂), 1.35-1.20 (m, 6H, CH₂), 0.83 (t, J=7.0 Hz, 3H, CH₃); ¹³C NMR (CDCl₃), δ 172.5 (7a-C), 160.5 (C-2), 156.4 (C-6), 139.5 (4-CH), 108.3 (4a-C), 98.6 (5-CH), 40.2 (N—CH₃), 31.8 (α-CH₂), 29.1 (CH₂), 28.7 (CH₂), 27.1 (CH₂), 22.9 (CH₂), 14.5 (CH₃).

### 2-Butyloxy-6-hexyl-furo[2,3-d]pyrimidine Cf2346

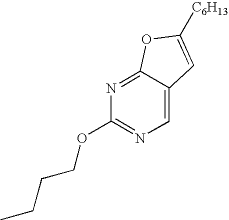

6-Hexyl-2,3-dihydrofuro[2,3-d]pyrimidin-2-one 38 (0.40 g, 1.82 mmol), potassium carbonate (0.50 g, 3.65 mmol, 2 equiv) and 1-iodobutane (0.41 mL, 3.62 mmol, 2 equiv) were suspended in dry DMF (5 mL) under N₂ and heated to 80° C. with stirring overnight. The solvents were removed in vacuo and the crude purified by flash column chromatography in a 0-5% MeOH/DCM solvent gradient to yield the title product 42 as a white solid (180 mg, 36%).

¹H NMR (CDCl₃) δ 8.65 (s, 1H, H-4), 6.34 (s, 1H, H—S), 4.42 (t, J=6.6 Hz, 2H, O—CH₂—), 2.77 (t, J=7.5 Hz, 2H, α-CH₂), 1.86 (qt, J=7.5 Hz, 2H, CH₂), 1.76 (qt, J=7.5 Hz, 2H, CH₂), 1.55 (m, 2H, CH₂), 1.43-1.31 (m, 6H, CH₂), 1.00 (t, J=7.2 Hz, 3H, CH₃), 0.92 (t, J=6.8 Hz, 3H, CH₃); ¹³C NMR (CDCl₃) δ 168.8 (7a-C), 162.8 (C-2), 159.1 (C-6), 150.8 (4-CH), 99.6 (5-CH), 68.1 (O—CH₂—), 31.9 (α-CH₂), 31.3 (CH₂), 31.2 (CH₂), 29.1 (CH₂), 28.8 (CH₂), 27.6 (CH₂), 22.9 (CH₂), 19.6 (CH₂), 14.5 (CH₃), 14.2 (CH₃).

### 2-Benzyloxy-6-hexyl-furo[2,3-d]pyrimidine Cf2348

6-Hexyl-2,3-dihydrofuro[2,3-d]pyrimidin-2-one (44, 0.40 g, 1.82 mmol) and potassium carbonate (0.50 g, 3.64 mmol, 2 equiv) were added under N₂ to dry DMF (5 mL), and the resultant suspension charged with benzyl chloride 43 (0.42 mL, 3.64 mmol, 2 equiv), then heated to 80° C. overnight. The solvents were removed in vacuo and the crude purified by flash column chromatography in a 0-5% MeOH/DCM eluent gradient to yield 39 mg (44, 7%) of the title compound as a white solid.

¹H NMR (CDCl₃) δ 8.65 (br, 1H, H-4), 7.57 (d, J=7.4 Hz, 2H, Ar—CH), 7.46-7.36 (m, 3H, Ar—CH), 6.38 (s, 1H, H-5), 5.53 (s, 2H, Ph-CH₂), 2.81 (t, J=7.6 Hz, 2H, α-CH₂), 1.79 (qt, J=7.4 Hz, 2H, CH₂), 1.47-1.33 (m, 6H, CH₂), 0.95 (t, J=6.8 Hz, 3H, CH₃); ¹³C NMR (CDCl₃) δ 168.8 (7a-C), 162.5 (C-2), 159.4 (C-6), 150.9 (4-CH), 137.0 (Ar—C), 128.8 (Ar—C), 128.4 (Ar—C), 128.3 (Ar—C), 113.9 (4-CH), 99.6 (Ar—C), 69.7 (O—CH₂-Ph), 31.9 (α-CH₂), 29.1 (CH₂), 28.7 (CH₂), 27.1 (CH₂), 23.0 (CH₂), 14.5 (CH₃).

### 3-Benzyl-6-hexyl-3H-furo[2,3-d]pyrimidin-2-one Cf2349

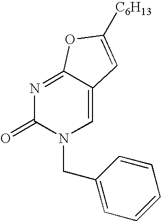

Also obtained from the purification process was the title compound 45 as a white solid (391 mg, 69%).

¹H NMR (CDCl₃) δ 7.89 (s, 1H, H-4), 7.49 (m, 5H, Ar—CH), 6.19 (s, 1H, H-5), 5.39 (s, 2H, Ph-CH₂), 2.76 (t, J=7.4 Hz, 2H, α-CH₂), 1.80 (qt, J=7.4 Hz, 2H, CH₂), 1.54-1.38 (m, 6H, CH₂), 1.02 (t, J=6.8 Hz, 3H, CH₃); ¹³C NMR (CDCl₃) δ 172.2 (7a-C), 160.7 (C-2), 156.1 (C-6), 138.5 (Ar—C), 136.0 (Ar—C), 129.5 (2×Ar—C), 129.0 (Ar—C), 128.9 (Ar—C), 108.6 (4-CH), 98.9 (5-CH), 54.5 (N—CH₂-Ph), 31.8 (α-CH₂), 29.1 (CH₂), 28.7 (CH₂), 27.1 (CH₂), 22.9 (CH₂), 14.5 (CH₃).

Biological Activity

Products where X═Y═N, Z=Q=O, U═V═CH and R¹, R⁴ and R⁸ are as given in Tables 1 and 2 below embodying the present invention were tested in vitro in tissue cultures for toxicity and for potent antiviral actions with respect to cytomegalovirus (CMV). The results are given in Tables 1 and 2 below.

The column headings in Tables 1 and 2 are as follows:

R¹, R⁴ and R⁸ are as defined with respect to formula I above.

EC₅₀/μm CMV-AD169 is the drug concentration in μM required to reduce by 50% CMV strain AD169 induced cytopathicity in human embryonic lung fibroblast (HEL) cells measured 7 days post infection compared to untreated control.

EC₅₀/μM CMV Davis is the drug concentration in μM required to reduce by 50% CMV strain Davis induced cytopathicity in human embryonic lung fibroblast (HEL) cells measured 7 days post infection compared to untreated control.

CC₅₀/μM is the compound concentration required to reduce the cell number by 50%.

Further details of the methodology employed can be found in McGuigan et al. J. Med. Chem., 1999, 42, 4479-4484.

TABLE 1

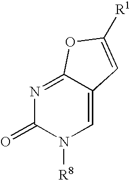

| | | | EC₅₀/μM | | |
|---|---|---|---|---|---|
| | | | CMV | 1 CMV | |
| No. | R¹ | R⁸ | AD169 | Davis | CC₅₀/μM |
| 2158 | nC₄H₉ | Cyclo C₅H₉ | >50 | >50 | ND |
| 2160 | nC₇H₁₅ | Cyclo C₅H₉ | 5 | 4 | 194 |
| 2194 | nC₄H₉ | CH(Et)₂ | >50 | >200 | >200 |
| 2190 | nC₇H₁₅ | CH(Et)₂ | 20 | 50 | >200 |
| 2195 | nC₄H₉ | nC₅H₁₁ | >50 | >50 | >200 |
| 2192 | nC₇H₁₅ | nC₅H₁₁ | >200 | >200 | >200 |
| 2196 | nC₇H₁₅ | 2-THF | >20 | >20 | 46 |
| 2249 | nC₁₀H₂₁ | 2-THF | >50 | 50 | >200 |
| 2275 | nC₁₀H₂₁ | CH₂Cyclo C₆H₁₁ | >200 | >200 | >200 |
| 2276 | nC₁₀H₂₁ | 3-THF | 20 | 10 | 148 |
| 2295 | nC₁₀H₂₁ | Cyclo C₆H₁₁ | 38 | 50 | >200 |
| 2304 | nC₁₀H₂₁ | C₃H₇ | 40 | 8 | >200 |
| 2306 | nC₁₀H₂₁ | nC₄H₉ | >200 | >200 | >200 |
| 2308 | nC₁₀H₂₁ | PhCH₂ | >40 | >40 | >200 |
| 2314 | nC₁₀H₂₁ | TolCH₂ | >40 | >40 | >200 |
| 2316 | nC₁₀H₂₁ | pMeOPhCH₂ | >200 | >200 | >200 |
| 2309 | nC₁₀H₂₁ | CH₂Cyclo C₅H₉ | 0.78 | 0.84 | 49 |
| 2344 | nC₆H₁₃ | Me | 18 | 20 | ND |
| 2345 | nC₆H₁₃ | nC₃H₇ | 20 | 20 | ND |
| 2347 | nC₆H₁₃ | nC₄H₉ | 19 | 20 | ND |
| 2349 | nC₆H₁₃ | PhCH₂ | >200 | >200 | ND |

TABLE 2

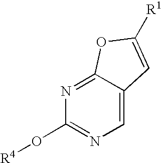

| | | | EC₅₀/μM | | |
|---|---|---|---|---|---|
| | | | CMV | 2 CMV | |
| No. | R¹ | R⁴ | AD169 | Davis | CC₅₀/μM |
| 2159 | nC₄H₉ | Cyclo C₅H₉ | 8 | 7 | 108 |
| 2161 | nC₇H₁₅ | Cyclo C₅H₉ | 3 | 5 | 132 |
| 2193 | nC₄H₉ | CH(Et)₂ | >20 | >20 | 98 |
| 2189 | nC₇H₁₅ | CH(Et)₂ | >5 | 12 | 98 |
| 2191 | nC₇H₁₅ | nC₅H₁₁ | >5 | 16 | 1109 |
| 2247 | nC₁₀H₂₁ | nC₅H₁₁ | >200 | >200 | >200 |
| 2250 | nC₁₀H₂₁ | Cyclo C₅H₉ | >50 | >50 | >200 |
| 2252 | nC₁₀H₂₁ | CH(Et)₂ | 16 | 10 | 127 |
| 2294 | nC₁₀H₂₁ | Cyclo C₆H₁₁ | 12 | 16 | >200 |
| 2303 | nC₁₀H₂₁ | nC₃H₇ | 2.5 | 2.1 | 126 |
| 2305 | nC₁₀H₂₁ | nC₄H₉ | 3.9 | 2.7 | >200 |
| 2307 | nC₁₀H₂₁ | PhCH₂ | 3.3 | 1.1 | >200 |
| 2274 | nC₁₀H₂₁ | CH₂CycloC₆H₁₁ | 4.4 | 2.9 | >200 |
| 2313 | nC₁₀H₂₁ | TolCH₂ | 10.5 | 3.9 | >200 |
| 2315 | nC₁₀H₂₁ | pMeOPhCH₂ | 3.3 | 2.9 | >200 |
| 2343 | nC₆H₁₃ | Me | >8 | 4.7 | ND |
| 2346 | nC₆H₁₃ | nC₄H₉ | 8 | 3 | ND |
| 2348 | nC₆H₁₃ | PhCH₂ | >200 | >3.6 | ND |

1. A chemical compound having the formula (I): 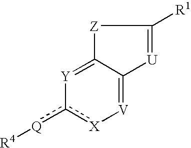
wherein:
R¹ and R⁴ are independently selected from alkyl, aryl, alkenyl and alkynyl;
Z is selected from O, NH, S, Se, NR¹ and (CH₂), where n is 1 to 10, and CT₂ where T may be the same or different and is selected from hydrogen, alkyl and halogens, and R⁵ is alkyl, alkenyl or aryl;
Y is selected from N, CH and CR⁶ where R⁶ is alkyl, alkenyl, alkynyl or aryl;
Q is selected from O, S, NH, N-alkyl, CH₂, CHalkyl and C(alkyl)₂;
U is selected from N and CR², R² is selected from hydrogen, alkyl, halogen, amino, alkylamino, dialkylamino, nitro, cyano, alkoxy, aryloxy, thiol, alkylthiol, arylthiol and aryl;
V is selected from N and CR³, where R³ is selected from hydrogen, alkyl, halogens, alkyloxy, aryloxy and aryl; and
when a double bond exists between X and the ring atom to which Q is attached and Q is linked to the ring moiety by a single bond, X is selected from N, CH and CR⁷, where R⁷ is selected from alkyl, alkenyl, alkynyl and aryl; and
when a double bond links Q to the ring moiety and a single bond exists between X and the ring atom to which Q is attached, R⁴ does not exist and X is NR⁸, where R⁸ is alkyl, alkenyl, alkynyl or aryl, except that when Y is N, R⁸ is not an alkyl or alkenyl group substituted at the fourth atom of the chain of said alkyl or alkenyl group, counted along the shortest route away from the ring moiety including any heteroatom present in said chain, by a member selected from OH, phosphate, diphosphate, triphosphate, phosphonate, diphosphonate, triphosphonate, and pharmacologically acceptable salts, derivatives and prodrugs thereof;
and pharmacologically acceptable salts, derivatives and prodrugs of compounds of formula I.

2. A compound according to claim 1 wherein when a double bond exists between X and the ring atom to which Q is attached, X and Y are both N.

3. A compound according to claim 1 wherein when a double bond exists between X and the ring atom to which Q is attached, Z is O or NH, preferably O.

4. A compound according to claim 1 wherein when a double bond exists between X and the ring atom to which Q is attached, Q is O.

5. A compound according to claim 1 wherein X and Y are N, Q and Z are independently selected from O, S and NH, and preferably both Q and Z are O.

6. A compound according to an) one of claims claim 1 toI wherein each of U and V is CH.

7. A compound according to claim 1 wherein R¹ is selected from C₃₋₂₀alkyl, C₃₋₂₀cycloalkyl, C₃₋₂₀alkenyl, C₃₋₂₀alkynyl, C₅₋₁₄aryl and C₁₋₁₀alkylC₅₋₁₄aryl, preferably C₃₋₁₄alkyl, C₃₋₁₄alkenyl and C₃₋₁₄alkynyl, more preferably C₈₋₁₀alkyl, C₈₋₁₀alkenyl and C₈₋₁₀alkynyl.

8. A compound according to claim 7 wherein R¹ is unbranched and unsubstituted C₃₋₁₂alkyl, preferably C₆₋₁₀alkyl.

9. A compound according to claim 1 wherein each of R⁴ and R⁸ is selected from C₁₋₁₂alkyl, C₁₋₁₂alkenyl, C₁₋₁₂alkynyl, C₃₋₁₂cycloalkyl, C₁₋₆alkyl substituted with C₃₋₇cycloalkyl, C₁₋₃alkyl, C₅₋₁₄aryl and C₃₋₆cycloalkyl and C₅₋₁₄aryl containing 1, 2, 3 or 4 hetero ring atoms independently selected form O, N and S, preferably R⁴ and R⁸ are selected from C₁₋₁₀alkyl, C₁₋₁₀alkenyl and C₁₋₁₀alkynyl.

10. A compound according to claim 1 wherein R¹ is C₃₋₁₄ alkyl, C₃₋₁₄ alkenyl or C₃₋₁₄ alkenyl, preferably C₆₋₁₄ alkyl, C₆₋₁₄ alkenyl or C₆₋₁₄ alkynyl, and R⁴ and R⁸ are selected from C₁₋₁₂ alkyl, C₃₋₁₀ cycloalkyl, C₁₋₆ alkyl substituted with C₃₋₇ cycloalkyl, preferably C₅₋₆ alkyl or C₅₋₆ cycloalkyl.

11. A compound according to claim 1 wherein R¹ is C₁₀ alkyl.

12. A compound according to claim 1 wherein R⁴ and R⁸ are selected from benzyl or substituted benzyl.

13. A compound according to claim 1 wherein R⁴ and R⁸ are C₁ alkyl substituted with C₁₋₁₀ cycloalkyl, preferably C₁ alkyl substituted with C₅₋₆ cycloalkyl.

14. A compound according to claim 1 wherein X and Y are both N, U and V are both CH, Z and Q are independently selected from O, S and NH, and each of R¹, R⁴ and R⁸ are C₈₋₁₂ alkyl.

15. A compound selected from the group comprising:
6-Butyl-3-cyclopentyl-3H-furo[2,3-d]pyrimidin-2-one (139) [Cf2158]
6-Butyl-2-cyclopentyloxy-furo[2,3-d]pyrimidine (130) [Cf2159]
6-Heptyl-3-cyclopentyl-3H-furo[2,3-d]pyrimidin-2-one (140) [Cf2160]
6-Heptyl-2-cyclopentyloxy-furo[2,3-d]pyrimidine (141) [Cf2161]
6-Butyl-3-(1-ethyl-propyl)-3H-furo[2,3-d]pyrimidin-2-one (142) [Cf2194]
6-Butyl-2-(1-ethyl-propoxy)-furo[2,3-d]pyrimidine (143) [Cf2193]
6-Heptyl-3-(1-ethyl-propyl)-3H-furo[2,3-d]pyrimidin-2-one (144) [Cf2190]
6-Heptyl-2-(1-ethyl-propoxy)-furo[2,3-d]pyrimidine (145) [Cf2189]
6-Butyl-3-pentyl-3H-furo[2,3-d]pyrimidin-2-one (146) [Cf2195]
6-Butyl-2-pentyloxy-furo[2,3-d]pyrimidine (147) [Cf2327]
6-Heptyl-3-pentyl-3H-furo[2,3-d]pyrimidin-2-one (148) [Cf2192]
6-Heptyl-3-pentyloxy-3H-furo[2,3-d]pyrimidin-2-one (149) [Cf2191]
6-Heptyl-3-(tetrahydro-furan-2-yl)-3H-furo[2,3-d]pyrimidin-2-one (154) [Cf2196]
6-Decyl-2-propoxy-furo[2,3-d]pyrimidine Cf2303
6-Decyl-3-propyl-3H-furo[2,3-d]pyrimidin-2-one Cf2304
2-Butoxy-6-decyl-furo[2,3-d]pyrimidine Cf2305
3-Butyl-6-decyl-3H-furo[2,3-d]pyrimidin-2-one Cf2306
6-Decyl-2-pentyloxy-2,3-dihydrofuro[2,3-d]pyrimidine Cf2247
2-Cyclopentyloxy-6-decyl-2,3-dihydrofuro[2,3-d]pyrimidine Cf2250
3-Cyclopentyl-6-decyl-2,3-dihydrofuro[2,3-d]pyrimidin-2-one Cf2251
2-(1′-Ethyl-propyloxy)-6-decyl-2,3-dihydrofuro[2,3-d]pyrimidine Cf2252
3-(1′-Ethyl-propyl)-6-decyl-2,3-dihydrofuro[2,3-d]pyrimidin-2-one Cf2253
2-Cyclohexyloxy-6-decyl-2,3-dihydrofuro[2,3-d]pyrimidine Cf2294
3-Cyclohexyl-6-decyl-2,3-dihydrofuro[2,3-d]pyrimidin-2-one Cf2295
6-Decyl-3-(tetrahydro-furan-2-ylmethyl)-3H-furo[2,3-d]pyrimidin-2-one 72 Cf2309
2-Cyclohexylmethoxy-6-decyl-furo[2,3-d]pyrimidine Cf2274
3-Cyclohexylmethyl-6-decyl-3H-furo[2,3-d]pyrimidin-2-one Cf2275
2-Benzyloxy-6-decyl-furo[2,3-d]pyrimidine Cf2307
3-Benzyl-6-decyl-3H-furo[2,3-d]pyrimidin-2-one Cf2308
6-Decyl-3-(tetrahydro-furan-2′-yl)-2,3-dihydrofuro[2,3-d]pyrimidin-2-one Cf2249
6-Decyl-2-(tetrahydro-furan-3-yloxy)-furo[2,3-d]pyrimidine 58
6-Decyl-3-(tetrahydro-furan-3-yl)-3H-furo]2,3-d]pyrimidin-2-one Cf2276
6-Decyl-2-(tetrahydro-furan-2-ylmethoxy)-furo[2,3-d]pyrimidine 71
6-Decyl-3-(tetrahydro-furan-2-ylmethyl)-3H-furo[2,3-d]pyrimidin-2-one 72
6-Decyl-2-(tetrahydro-pyran-2-ylmethoxy)-furo[2,3-d]pyrimidine 61
6-Decyl-3-(tetrahydro-pyran-2-ylmethyl)-3H-furo[2,3-d]pyrimidin-2-one 62
6-Decyl-2-(4-methoxybenzyloxy)-3H-furo[2,3-d]pyrimidine Cf2315
6-Decyl-3-(4-methoxybenzyl)-3H-furo[2,3-d]pyrimidin-2-one Cf2316
6-Decyl-2-(4-methylbenzyloxy)-3H-furo[2,3-d]pyrimidine Cf2313
6-Decyl-3-(4-methylbenzyl)-3H-furo[2,3-d]pyrimidin-2-one Cf2314
6-Hexyl-3-methyl-3H-furo[2,3-d]pyrimidin-2-one Cf2344
2-Butyloxy-6-hexyl-furo[2,3-d]pyrimidine Cf2346
2-Benzyloxy-6-hexyl-furo[2,3-d]pyrimidine Cf2348
3-Benzyl-6-hexyl-3H-furo[2,3-d]pyrimidin-2-one Cf2349.

16. A method for preparing compounds according to claim 1 wherein a 5-halo nucleoside analogue is contacted with a terminal alkyne in the presence of a catalyst, or a 5-alkynyl nucleoside is cyclised in the presence of a catalyst.

17. A compound according to claim 1 for use in a method of treatment.

18. Use of a compound according to claim 1 in the manufacture of a medicament for the prophylaxis or treatment of viral infection.

19. Use according to claim 18 wherein the viral infection is a cytomegalovirus viral infection.

20. A method of prophylaxis or treatment of viral infection comprising administration to a patient in need of such treatment an effective dose of a compound according to claim 1.

21. A method according to claim 20 wherein the viral infection is a cytomegalovirus viral infection.

22. A compound according to claim 1 in the manufacture of a medicament for use in the prophylaxis or treatment of a viral infection.

23. A compound according to claim 22 wherein the viral infection is a cytomegalovirus viral infection.

24. A pharmaceutical composition comprising a compound according to claim 1 in combination with a pharmaceutically acceptable excipient.

25. A method of preparing a pharmaceutical composition comprising the step of combining a compound according to claim 1 with a pharmaceutically acceptable excipient.
